(19) United States
(12) Patent Application Publication
(10) Pub. No.: US 2025/0375198 A1
(43) Pub. Date: Dec. 11, 2025

(54) SUTURES AND DELIVERY ELEMENTS OF REMOTE SURGICAL SUTURE SYSTEMS AND DEVICES, AND METHODS THEREOF

(71) Applicant: Vesteck, Inc., West Chester, PA (US)

(72) Inventors: Edward WULFMAN, Woodinville, WA (US); Kent STALKER, San Marcos, CA (US); John WHITFIELD, Temecula, CA (US); John K. EDOGA, New York, NY (US); Thierry RICHARD, Villiers Le Mahieu (FR); Kenneth STALKER, San Marcos, CA (US)

(21) Appl. No.: 19/017,031

(22) Filed: Jan. 10, 2025

Related U.S. Application Data
(63) Continuation of application No. PCT/US2023/070175, filed on Jul. 13, 2023.
(60) Provisional application No. 63/389,157, filed on Jul. 14, 2022.

(51) Int. Cl. A61B 17/04; A61B 17/00; A61B 90/00

(52) U.S. Cl. CPC A61B 17/0487; A61B 17/0401; A61B 90/39; A61B 2017/00526; A61B 2017/00862; A61B 2017/0461; A61B 2017/0488; A61B 2090/3966

(57) ABSTRACT

Systems, devices and methods described herein relate to sutures and endovascular devices for delivering same. In some embodiments, a suture includes a set of legs having proximal ends joined to each other at a bridge and elongate bodies that extend from the bridge. In some embodiments, the suture can include a bend, a protective element, or three or more legs.

### CROSS-REFERENCE TO RELATED APPLICATIONS

This application is a continuation application of PCT International Patent Application No. PCT/US2023/070175, filed on Jul. 13, 2023, titled “SUTURES AND DELIVERY ELEMENTS OF REMOTE SURGICAL SUTURE SYSTEMS AND DEVICES, AND METHODS THEREOF,” which claims priority to U.S. Provisional Patent Application No. 63/389,157, filed Jul. 14, 2022, titled “SUTURES AND DELIVERY ELEMENTS OF REMOTE SURGICAL SUTURE SYSTEMS AND DEVICES, AND METHODS THEREOF,” the contents of which are incorporated by reference herein in their entirety.

### TECHNICAL FIELD

The present disclosure generally relates to endovascular devices and methods thereof, and in particular to endovascular suture delivery systems and methods of treating vascular disease.

### BACKGROUND

Remote surgical and interventional procedures are often done to avoid the trauma and potential complications of open surgery. An example is percutaneously placed grafts for treatment of abdominal aortic aneurysms (AAA), or AAA endovascular repair, which can avoid the major trauma of open surgical aortic repair. Remote surgery and interventional procedures can be done on major organs including heart, liver, kidneys, and the like. Other examples of minimally invasive or remote surgery include minimally invasive gastrointestinal and musculoskeletal surgeries.

Open surgery, however, can provide better long-term outcomes, partly because surgeons can use sutures in a secure manner to attach grafts and other materials to patient vessels to achieve a desired long-term result. Remote surgical and interventional procedures may not be able to achieve placement of sutures in a manner that mimics the secure sutures surgeons place when performing open surgery. The AAA grafts mentioned above are an example where surgically tied sutures ensure the graft and the aorta do not move relative to another as the patient ages and the body changes. Complications from endovascular grafts which are not typically sutured in place can include endovascular leaks, increased risk of aneurysm failures, and the like. Similar examples exist for minimally invasive remote surgery as compared to open surgery in other areas of the body such as those listed above, oftentimes for the same reason of not being able to place secure sutures.

Some examples exist of devices that intend to hold a graft in place. One example is an anchoring mechanism that can be delivered percutaneously to a location of a graft and used to anchor the graft to a wall of the aorta. For example, U.S. Pat. No. 8,157,146, titled “Stapling Device,” issued Apr. 17, 2012, and U.S. Pat. No. 8,627,992, titled “Endovascular Stapler,” issued Jan. 14, 2014, describe examples of staple delivery devices for delivering staples for fixation with a graft and aorta. Such devices, however, can be limited in their ability to deliver multiple anchoring mechanisms or staples and in delivering anchoring mechanisms or staples that clamp the graft and the aorta together to prevent the mechanisms from backing out.

Thus, there is a need for systems, devices, and methods that can be placed in a percutaneous procedure, that can securely hold and clamp, and can deliver multiple sutures.

### SUMMARY

Systems, devices and methods described herein relate to endovascular devices for delivering sutures for securing grafts or other objects to patient vessels. In some embodiments, a suture includes: a set of two legs having proximal ends joined to each other and elongate bodies that extend parallel to one another such that the set of two legs form a U-shaped structure, the set of legs having distal ends that terminate in sharpened tips that are configured to penetrate through a portion of a graft and a vessel wall adjacent to the portion of the graft; a tail coupled to the U-shaped structure, the suture configured to transition from a flattened configuration in which the tail extends in a direction opposite to the direction of the elongate bodies of the two legs to a curved configuration in which the tail and the set of two legs are curved, the set of legs and the tail configured to exert forces in opposite directions when the suture is in the curved configuration such that the set of legs and the tail are configured to hold the portion of the graft and the vessel wall relative to one another.

In some embodiments, a suture delivery system, includes: a housing configured to contain a suture in a flattened configuration, the housing defining an opening for releasing the suture from an interior of the housing such that the suture can automatically transition from the flattened configuration into a curved configuration; a deployment element having a ribbon-shaped distal portion disposed in the housing, the ribbon-shaped distal portion of the deployment element having a surface with a set of formations configured to interface with the suture, the housing and the set of formations of the deployment element configured to collectively constrain the suture in the flattened configuration until the suture is released through the opening of the housing; and an actuator configured to move the deployment element relative to the housing to release the suture from the opening of the housing.

In some embodiments, a suture delivery system includes: a housing configured to contain a suture in a flattened configuration, the housing defining an opening for releasing the suture from an interior of the housing such that the suture can automatically transition from the flattened configuration into a curved configuration, the housing at least partially constraining the suture in the flattened configuration until the suture is released through the opening of the housing; a deployment element having a distal portion disposed in the housing, the distal portion of the deployment element having one or more formations configured to interface with the suture; a biasing mechanism disposed about a shaft, the biasing mechanism configured to transition between an undeployed configuration in which the biasing mechanism extends generally parallel to the housing and the distal portion of the deployment element and an expanded configuration in which a portion of the biasing mechanism bows outward in a direction away from the housing to form an asymmetrical shape that presses the housing against a suture site; and an actuator configured to move the deployment element relative to the housing when the biasing mechanism is in the deployed configuration to release the suture from the housing to deploy the suture in the suture site.

In some embodiments, a suture delivery method includes: manipulating a first control mechanism of a suture delivery system to expand a biasing mechanism of the suture delivery system, the biasing mechanism when expanded configured to press a housing of the suture delivery system against a portion of a graft disposed in a vessel, the housing configured to constrain a suture in a flattened configuration and including an opening through which the suture can be deployed from the housing such that the suture can transition to a natural curved configuration; manipulating a second control mechanism of the suture delivery system one or more times to cause a deployment element disposed within the housing to move proximally to partially deploy the suture from the housing such that a set of legs of the suture exit the opening of the housing and curve to penetrate through the portion of the graft and the vessel; and manipulating the second control mechanism one or more additional times to cause the deployment element to continue to move proximally to fully deploy the suture from the housing such that a tail of the suture that extends opposite to the legs exit the opening of the housing and curve to press against material disposed between the set of legs of the suture to hold the graft and the vessel relative to one another.

In some embodiments, a suture delivery method includes: manipulating a first control mechanism of a suture delivery system to expand a biasing mechanism of the suture delivery system, the biasing mechanism when expanded configured to press a housing of the suture delivery system against a portion of a graft disposed in a vessel, the housing configured to constrain a suture in a flattened configuration and including an opening through which the suture can be deployed from the housing such that the suture can transition to a natural curved configuration; with a mode selector of the suture delivery system in a first position such that a second control mechanism of the suture delivery system is engaged with a drive system of the suture delivery system, manipulating the second control mechanism one or more times to cause a deployment element disposed within the housing to move proximally to partially deploy the suture from the housing; in response to the mode selector being set to a second position, disengaging the second control mechanism from the drive system; and moving the deployment element distally to retract the suture back into the housing.

In some embodiments, a suture includes: a set of legs having proximal ends joined to each other and elongate bodies that extend parallel to one another such that the set of legs form a U-shaped structure, the set of legs having distal ends that terminate in sharpened tips that are configured to penetrate through a portion of a graft and a vessel wall adjacent to the portion of the graft; a tail having a proximal end and a distal end, the distal end being coupled to the U-shaped structure; a clamp head coupled to the proximal end of the tail; and an engagement element disposed between the U-shaped structure and the clamp head, the engagement element configured to provide a surface for engaging with a suture advancement element to deploy the suture, the suture configured to transition from a flattened configuration in which the tail extends in a direction opposite to the direction of the elongate bodies of the legs to a curved configuration in which the tail and the set of legs are curved, the set of legs and the tail configured to exert forces in opposite directions when the suture is in the curved configuration such that the set of legs and the tail are configured to hold the portion of the graft and the vessel wall relative to one another.

In some embodiments, a suture includes: a single leg having an elongate body, the single leg having a distal end that terminates in a sharpened tip that is configured to penetrate through a portion of a graft and a vessel wall adjacent to the portion of the graft; a clamp having a proximal end and a distal end, the distal end being coupled to the single leg; and a clamp head coupled to the proximal end of the clamp, the suture configured to transition from a flattened configuration in which the clamp extends in a direction opposite to the direction of the elongate body of the single leg to a curved configuration in which the clamp and the single leg are curved, the single leg and the clamp configured to exert forces in opposite directions when the suture is in the curved configuration such that the single leg and the clamp are configured to hold the portion of the graft and the vessel wall relative to one another.

In some embodiments, a suture includes: a bridge; a set of legs having proximal ends joined to the bridge and elongate bodies that extend away from the bridge, the elongate bodies being coupled to or including a set of bends, the set of legs having distal ends that terminate in sharpened tips that are configured to penetrate through a portion of a graft and a vessel wall adjacent to the portion of the graft; a tail having a proximal end and a distal end, the distal end being coupled to the bridge; and a clamp head coupled to the proximal end of the tail; the suture configured to transition from a flattened configuration in which the tail extends in a direction opposite to the direction of the elongate bodies of the legs to a curved configuration in which the tail and the set of legs are curved, the set of legs and the tail configured to exert forces in opposite directions when the suture is in the curved configuration such that the set of legs and the tail are configured to hold the portion of the graft and the vessel wall relative to one another.

In some embodiments, a suture includes: a bridge; a set of legs having proximal ends joined to the bridge and elongate bodies that extend away from the bridge, the set of legs having distal ends that terminate in sharpened tips that are configured to penetrate through a portion of a graft and a vessel wall adjacent to the portion of the graft; a tail having a proximal end and a distal end, the distal end being coupled to the bridge; and a clamp head coupled to the proximal end of the tail, the clamp head having a set of edges configured to direct external devices toward a proximal end of the clamp head; the suture configured to transition from a flattened configuration in which the tail extends in a direction opposite to the direction of the elongate bodies of the legs to a curved configuration in which the tail and the set of legs are curved, the set of legs and the tail configured to exert forces in opposite directions when the suture is in the curved configuration such that the set of legs and the tail are configured to hold the portion of the graft and the vessel wall relative to one another.

### BRIEF DESCRIPTION OF DRAWINGS

FIG. 1 is a schematic block diagram of a suture delivery system for endovascularly delivering sutures, according to an embodiment.

FIG. 2 is a schematic diagram of a catheter assembly of a suture delivery system, according to an embodiment.

FIG. 3 is a schematic diagram of a handle assembly of a suture delivery system, according to an embodiment.

FIG. 4 is a schematic diagram of a suture, according to an embodiment.

FIGS. 5A-5B schematically depict a catheter assembly of a suture delivery system in different configurations, according to an embodiment.

FIG. 6A is a side view of an example suture delivery system for endovascularly delivering sutures, according to an embodiment.

FIG. 6B is a detailed view of a distal portion of an example suture delivery system, according to an embodiment.

FIG. 7A is a side view of an example suture delivery system having an expanded biasing mechanism, according to an embodiment.

FIG. 7B is a detailed view of a distal portion of an example suture delivery system having an expanded biasing mechanism, according to an embodiment.

FIGS. 8A-8B are side and cross-sectional views, respectively, of a handle of a suture delivery system, according to an embodiment.

FIG. 9 a schematic diagram of a suture delivery system showing connections between proximal handle components and distal catheter components, according to an embodiment.

FIG. 10 is a perspective view of a suture advancement and retraction element of a suture delivery system, according to an embodiment.

FIG. 11 is a detailed view of a distal portion of a suture advancement and retraction element of a suture delivery system, according to an embodiment.

FIGS. 12A-12C are different views of an example suture that can be used with the suture advancement and retraction element depicted in FIGS. 10-11, according to an embodiment.

FIG. 13 is an example of a suture advancement and retraction element with a suture disposed thereon, according to embodiments.

FIGS. 14A-14F depict a progression of releasing a suture from a suture delivery system where the suture is released from a side of the system, according to an embodiment.

FIG. 15A illustrates different views of a suture with angled tips in a flat configuration, according to an embodiment.

FIG. 15B is a black and white photograph of a suture with angled tips in a curved configuration, according to an embodiment.

FIGS. 16A-16B are black and white photographs show manufacturing of angled or sharpened tips of sutures, according to various embodiments.

FIG. 17A is an illustration of a suture with angled tips and an engagement portion, in a flat configuration, according to an embodiment.

FIG. 17B is a photograph of a suture with angled tips and an engagement portion, in a curved configuration, according to an embodiment.

FIG. 18A is an illustration of a suture with angled tips and an engagement portion, in a partially flattened configuration, according to an embodiment.

FIGS. 18B-18C are illustrations of a suture with angled tips and an engagement portion, the suture engaged with a suture advancement/retraction element, according to an embodiment.

FIG. 19A is an illustration of a suture with a single leg and a positive deployment feature, in a flat configuration, according to an embodiment.

FIG. 19B is an illustration of a suture with a single leg and a positive deployment feature, in a curved configuration, according to an embodiment.

FIG. 20A is an illustration of a suture with two legs and a positive deployment feature, in a flat configuration, according to an embodiment.

FIG. 20B is an illustration of a suture with two legs and a positive deployment feature, in a curved configuration, according to an embodiment.

FIGS. 21A-21B are illustrations of a suture with a radiopaque marker, according to an embodiment.

FIG. 22A is an illustration of a suture and a suture housing with radiopaque marker bands, according to an embodiment.

FIG. 22B is an X-ray image of a suture advancement/retraction element with radiopaque marker bands, according to an embodiment.

FIGS. 23A-23B show a manufacturing process of a shim of a suture housing, according to an embodiment.

FIG. 24A is a schematic diagram of a suture, according to an embodiment.

FIG. 24B is a schematic diagram of a suture, according to an embodiment.

FIG. 25A is an illustration of a suture with two legs having curves added orthogonal to the suture radius, in a curved configuration, according to an embodiment.

FIG. 25B is an illustration of a suture with two legs having curves added orthogonal to the suture radius, in a flat configuration, according to an embodiment.

FIG. 26 is an illustration of a suture with an angled clamp head, in a curved configuration, according to an embodiment.

FIG. 27 is an illustration of a suture with an arrowhead-shaped clamp head, in a curved configuration, according to an embodiment.

FIGS. 28A-28C are illustrations of a suture with a protective shield, according to an embodiment.

FIG. 29 is an illustration of a suture with three legs, in a curved configuration, according to an embodiment.

FIGS. 30A-30B are flow charts of an example process for placing a suture, according to an embodiment.

### DETAILED DESCRIPTION

Described herein are systems, devices, and methods for delivering sutures. In some embodiments, the systems, devices, and methods described herein may be used to bind together (e.g., to suture) a portion of an aorta and a graft, placing sutures during an endoscopic sleeve gastroplasty, during laparoscopic or robotic hernia repair, or during any other minimally invasive surgery procedures.

In some embodiments, the device described in this disclosure is an interventional medical device designed to deliver sutures to secure together a graft and a vessel wall. In some cases, a graft may be formed from a body tissue of a patient and in other cases, the graft may be engineered from other tissues (e.g., plastic, fabric, or other suitable soft and flexible tissue that may be penetrated by a suture). In an example embodiment an endovascular graft may be secured to the aorta for AAA repair. For instance, an example procedure for AAA repair generally involves delivering a graft to the inside of an aorta, and then securing the graft in place to form a stable channel for blood flow. After a graft is in place, the disclosed device can be used to deliver multiple sutures to the site of the graft to secure the graft to the neighboring vessel wall.

Suitable examples of suture delivery systems are described in International PCT Patent Application No. PCT/US2021/048888, published as PCT Publication No. WO 2022/051512, filed Sep. 2, 2021, and titled “Remote Surgical Suture System,” the disclosure of which is incorporated herein by reference. Further details of suture delivery systems are described in the following paragraphs. It can be appreciated that any of the suture delivery systems and/or elements thereof described below can incorporate one or more elements of the suture delivery systems described in International PCT Patent Application No. PCT/US2021/048888.

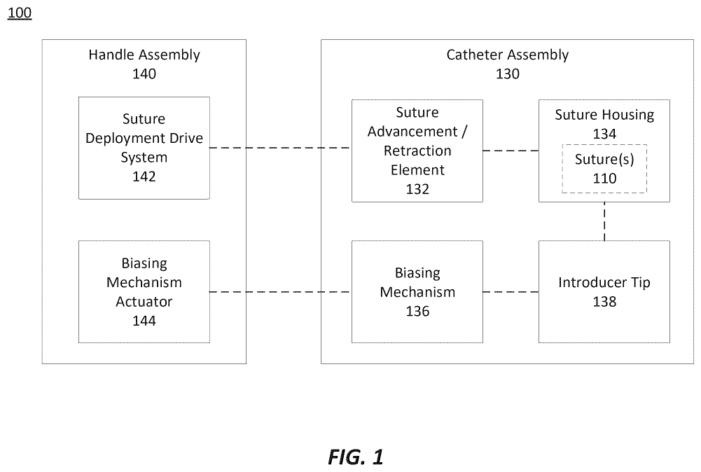

FIG. 1 is a schematic view of an example suture delivery system 100 for placing sutures 110 in a body of a patient, according to embodiments. Sutures 110 can be delivered into a portion of a patient's body via a catheter assembly 130. In some embodiments, a distal portion of the catheter assembly 130 may be guided to a region of a body that requires suturing (e.g., to the region in which a graft needs to be secured to a vessel wall such as the aorta of a patient. The distal portion of the catheter assembly 130 can be guided via a guidewire. For example, the catheter assembly 130 can define a guidewire lumen (not depicted) that can receive a guidewire and be guided along the guidewire to a suture site. Alternatively or additionally, the distal portion of the catheter assembly 130 can be guided via an access or introducer sheath to a suture site. For example, an access sheath may have been placed within the patient's vasculature, and the distal portion of the catheter assembly 130 can be advanced through the access sheath to the suture site. The distal portion of the catheter assembly 130 can be configured to release sutures at the suture site or region that requires suturing to bind or secure tissues and/or other material together (e.g., the aorta and the graft). As illustrated in FIG. 1, catheter assembly 130 can include a suture housing 134 configured to contain sutures 110. Suture housing 134 forms an enclosure that can constrain the sutures 110 inside (e.g., in a first state such as a flattened state, as further described below) and may include a suitable deployment window or opening for releasing sutures 110. The enclosure of suture housing 134 may have any suitable form for containing the sutures 110. In some embodiments, the suture housing 134 can have an elongate shape and can have a cross-sectional area that is or is substantially rectangular, square, trapezoidal, circular, etc.

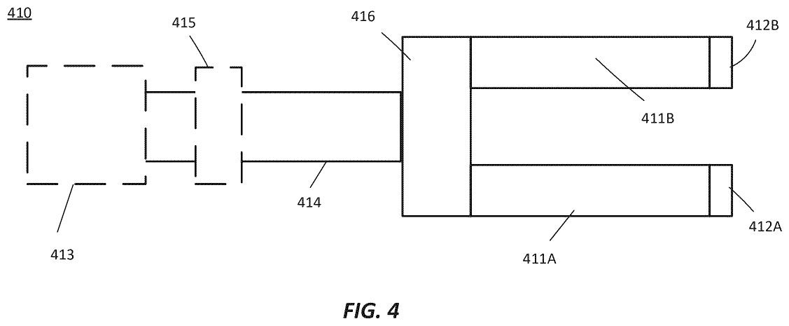

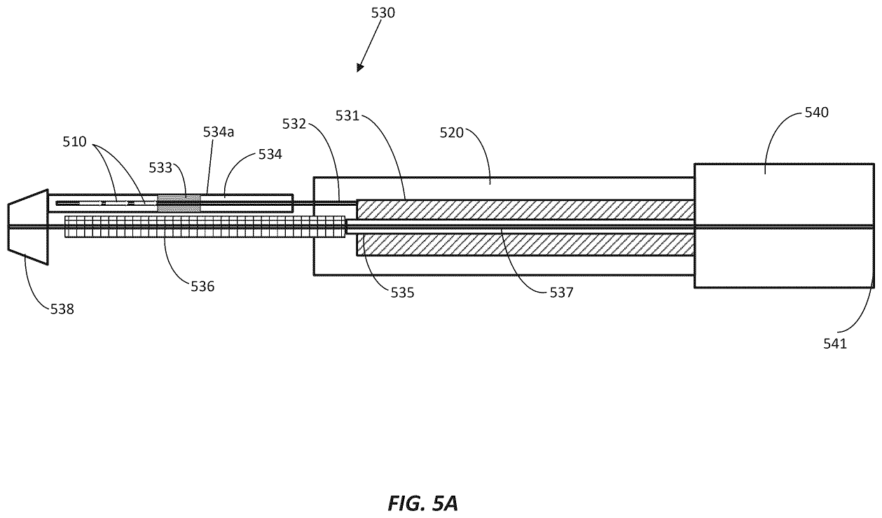

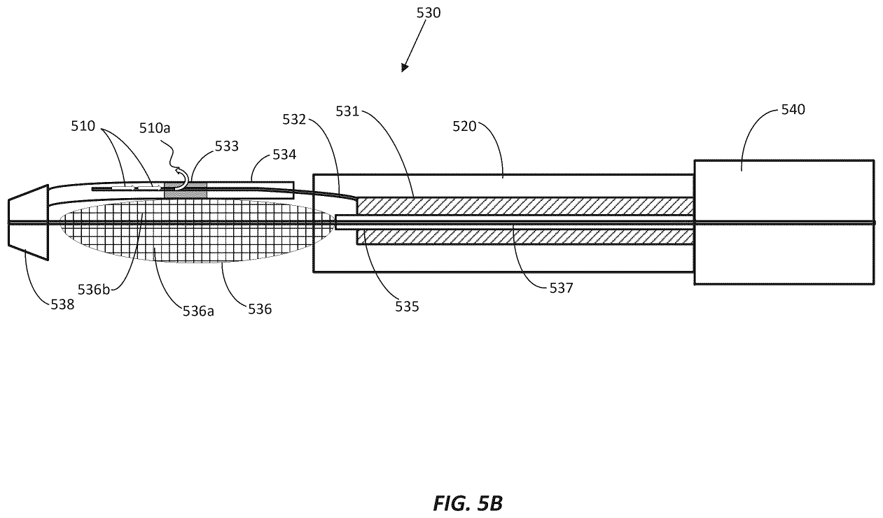

Sutures 110 can be configured to clamp tissue and other materials together. For example, sutures 110 can include different portions that exert opposing forces that clamp together tissue and other materials or create a tightening effect on such tissue and other materials. In various embodiments, sutures 110 are configured to change shape (e.g., bend and/or fold) when being released from suture housing 134. In other words, sutures 110 can be configured to transition from a first configuration to a second configuration when the suture is released from the suture housing 134. The first configuration can be a constrained configuration, such as, for example, a flattened configuration. The second configuration can be a natural or deployed configuration, such as, for example, a curved configuration. In some cases, sutures 110 may be configured to automatically or spontaneously transition into the deployed configuration when sutures 110 are released from suture housing 134. For example, sutures 110 may be formed of shape-memory material or superelastic material and can be configured to revert back to a natural state (e.g., a curved state) while being released from the suture housing 134. Alternatively, in some cases, forces and/or torques may be exerted on sutures 110 to facilitate or cause the transition of the sutures 110 into their deployed configuration. Example shape-memory or superelastic materials may be metals, such as Nickel-Titanium alloy (nitinol), stainless steel, Elgiloy or cobalt alloy, plastic, or other materials known in the art that has the desired flexibility and strength to be flattened and can reform to the desired state and effectively clamp different materials or tissue together when released from suture housing 134. In example embodiments, nitinol50 (Ni_{0.5}Ti_{0.5}), nitinol60 (Ni_{0.4}Ti_{0.6}) or other compositions of nitinol described by Ni₁₋ₓTiₓ may be used. In some cases, sutures 110 may be made from plastic, metal, or a combination of plastic and/or metal materials. In some embodiments, sutures 110 may include coatings, such as radiopaque coatings, or markers, as further described below. Various embodiments of sutures 110 are further described below with reference to FIGS. 4-5E.

Catheter assembly 130 further includes a suture advancement/retraction element (SARE) or suture deployment element 132. As part of suture advancement, SARE 132 is configured to release sutures 110 from suture housing 134. In some embodiments, SARE 132 is configured to release each of sutures 110 by moving a suture toward a deployment window (e.g., an opening) in suture housing 134 and pushing the suture through the deployment window. As a part of suture retraction, SARE 132 is configured to retract a suture that is partially released back into the suture housing 134. In some embodiments, SARE 132 may retract the suture by moving the suture away from the deployment window of suture housing 134. In some embodiments, SARE 132 can be configured to move the sutures 134 by sliding (e.g., advancing and retracting) within the suture housing 134. For example, a distal portion of the SARE 132 can be disposed within the housing 134 and be configured to slide or move relative to the housing to move the sutures 110. Further details of advancing and retracting sutures 110 from suture housing 134 using SARE 132 are discussed below. Additionally, various embodiments of SARE 132 are also discussed below.

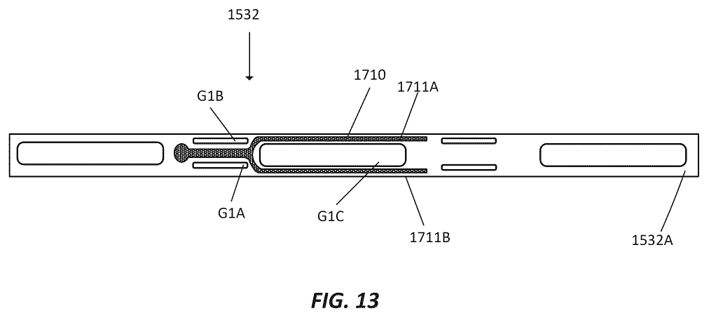

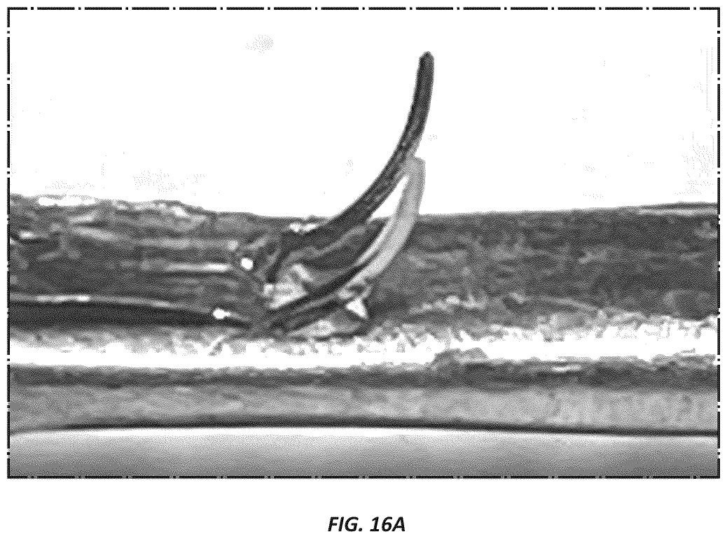

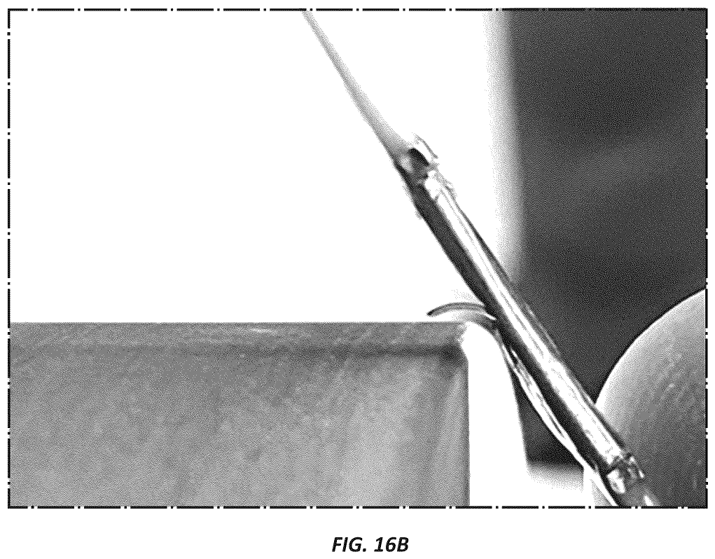

In some embodiments, SARE 132 can be implemented as an elongate structure with at least a region that has a flattened shape. For example, SARE 132 can have a ribbon-shaped structure or have a portion for receiving sutures 110 (e.g., a distal portion) that has a ribbon-shaped structure. In some embodiments SARE 132 can be implemented as a sled, e.g., an elongate strip having a rectangular cross-sectional area. The SARE 132 can include a plurality of formations that are configured to receive respective portions of the sutures 134. In some embodiments, the plurality of formations can have a repeating pattern such that subsets or formations are configured to receive an individual suture. In some embodiments, the plurality of formations can include ridges, notches, groove, channels, canals, or any other suitable structure for receiving and interfacing with a portion of a suture 110. In some embodiments, the sutures 110 can be disposed serially (e.g., in a row) along a length of a portion of the SARE 132, e.g., along a distal portion of the SARE 132 where the plurality of formations are disposed. The SARE 132 and the suture housing 134 collectively can be configured to constrain the sutures 110 in a first configuration (e.g., a flattened configuration). In some embodiments, the SARE 132 can be configured to interface with each suture 110 along its entire length, e.g., to minimize or reduce natural curving or warping of the suture 110 back to a natural curved state. As can be appreciated, sutures 110 that are memory set to a curved configuration but are constrained in a flattened state within the suture housing 134 can have a tendency to revert back to their curved configuration (e.g., via twisting or warping). As such, the SARE 132 can be designed with formations that are configured to receive and radially constrain portions of the sutures to reduce unintended movement of the sutures, thereby ensuring that they exit the suture housing 134 substantially normal to a deployment window of the housing 134. Further details of the radial constraining of sutures are provided with reference to FIGS. 13 and 16.

Catheter assembly 130 further includes an introducer tip 138 located at the distal end of catheter assembly 130. Introducer tip 138 may be an atraumatic structure (e.g., having a substantially conical, spherical, or other atraumatic shape) configured to facilitate introduction and navigation of catheter assembly 130 through patient vasculature to a region of the patient that requires suturing. In some embodiments, the introducer tip 138 can be coupled to a distal end of the suture housing 134, while a distal end of the SARE 132 can be free floating within the housing 134. As such, the SARE 132 can be configured to move or slide relative to the housing 134 when the introducer tip 138, the housing 134, and/or other components of the catheter assembly 130 are anchored or held in place within the patient, e.g., via a biasing mechanism 136 as further described below.

Catheter assembly 130 also includes a biasing mechanism 136. Biasing mechanism 136 is configured to change shape (e.g., expand). In other words, biasing mechanism 136 can be configured to transition from a first undeployed configuration in which the biasing mechanism 136 can extend generally or substantially parallel to a longitudinal axis of the catheter assembly 130 to a second deployed or expanded configuration in which the biasing mechanism 136 bows outward from the longitudinal axis. In some embodiments, the biasing mechanism 136 can be deployed to press a material, such as, for example, a graft, against a portion of tissue, such as, for example, a vessel wall (e.g., aortic wall). The biasing mechanism 136 can be configured to press a portion of the housing 134 containing the sutures 110 against the graft and tissue wall such that a window of the housing 134 through which the sutures 110 can be deployed is pressed against the graft. Such placement of the housing 134 and the window for deploying the sutures 110 can enable the suture delivery system 110 to deploy or deliver sutures through the window such that they directly contact and can penetrate through the graft and vessel wall. Further details of the sutures as they are deployed, and how the sutures can generate clamping or tightening effects on the tissue, are described with reference to later figures.

In an example embodiment, biasing mechanism 136 may be inserted into a vessel or a body cavity (e.g., in an aorta, a graft, or any other suitable vessel or body cavity) and may be expanded such that the biasing mechanism 136 directly contacts a portion of a material for suturing. The biasing mechanism in its expanded configuration can be configured to press against the walls of the vessel or body cavity to secure itself and other components coupled to it in place within the patient anatomy. Such securing or anchoring can ensure that a distal portion of the catheter assembly 130 and/or various components at the distal portion of the catheter assembly 130 do not move during a suturing procedure. For example, the biasing mechanism 136 can be coupled to the introducer tip 138, which can also be coupled to the suture housing 134. As such, expansion of the biasing mechanism 136 can maintain the introducer tip 136 and the suture housing 134 in place relative to a graft or vessel wall. The SARE 134 can then move (e.g., slide) within the suture housing 134 to deploy sutures out of the suture housing 134. In some cases, the biasing mechanism 136 can be undeployed (e.g., reverted back to its unexpanded state) to allow the distal portion of the catheter assembly 130 to move relative to the patient anatomy. In some embodiments, the biasing mechanism 136 can be implemented as an expandable mesh or basket formed of a plurality of wires. In some embodiments, the plurality of wires can be formed of a metallic material such as stainless steel, while in other embodiments, the plurality of wires can be formed of a flexible polymer or plastic. In some embodiments, the biasing mechanism 136 can be implemented as a balloon. In some embodiments, the biasing mechanism 136 can be deployed by moving an inner or other shaft relative to the other. For example, a distal end of the biasing mechanism 136 can be coupled to an inner shaft, and a proximal end of the biasing mechanism 136 can be coupled to an outer shaft. Movement of the inner or outer shaft relative to the other can then deploy the biasing mechanism 136, i.e., transition the biasing mechanism from its unexpanded configuration to its expanded configuration. In some embodiments, markings can be provided at a proximal end of the suture deliver system 100 (e.g., in handle assembly 140) that provides guidance to a surgeon on the degree or extent of expansion of the biasing mechanism 136. In some embodiments, biasing mechanism 136 can be configured to expand to a structure having a maximum diameter of between about 5 mm to about 60 mm, including all values and subranges therebetween. Other details and embodiments of biasing mechanism 136 are further discussed below.

According to various embodiments, and consistent with the disclosed embodiment shown in FIG. 1, various operations of catheter assembly 130 are controlled by a medical professional (e.g., a surgeon) through a use of a handle assembly 140. For example, the surgeon may operate elements of handle assembly 140 to deploy and/or retract sutures 110 via SARE 132, to deploy, partially deploy, or contract biasing mechanism 136, or to move catheter assembly 130 to a new position within a body of a patient. In some cases, operations of catheter assembly 130 guided by a surgeon operating handle assembly 140 may be based on imaging obtained during a surgical procedure. In an example embodiment, the imaging may be used to determine whether the sutures 110 are being properly placed through graft and/or tissue within a patient or to observe any relevant events that may influence the surgical procedure (e.g., internal bleeding, incorrect folding of tissue, a rupture of a tissue, and the like). Any suitable imaging may be used during the surgical procedure. For example, ultrasound imaging, imaging using a computed tomography (CT) system, or imaging using internally placed cameras or optics can be used. While not depicted, in some embodiments, the system 110 can include imaging devices that are positioned to capture images or video of the deployment of the biasing mechanism and/or deployment of individual sutures.

Handle assembly 140 includes a suture deployment drive system (SDDS) 142. In some embodiments, SDDS 142 can include a suitable mechanism for moving SARE 132. In an example embodiment, the suitable mechanism can be a deployment lever (DL) operatively coupled with SARE 132. A motion of the DL may control a motion of SARE 132, and, as a result, control the deployment and/or retraction of sutures 110 to or from tissues located within a patient. In some embodiments, SDDS 142 can include a button or other control mechanism that can be manipulated (e.g., depressed, slid, or otherwise moved) to move the SARE 132. In some embodiments, SDDS 142 can be coupled to an outer housing of the handle assembly 140, and the outer housing of the handle assembly 140 can be coupled to the SARE 132, e.g., via one or more connecting elements (e.g., shafts, fasteners, joints, etc.). Various embodiments of deployment drive system 142 are further described below.

Handle assembly 140 further includes a biasing mechanism actuator 144 configured to control expansion/contraction of biasing mechanism 136. In an example embodiment, biasing mechanism actuator 144 may include a slider or other suitable component (e.g., button, wheel, etc.) that can be moved (e.g., slid) to deploy the biasing mechanism 136. In some embodiments, the handle assembly 140 can include markings indicating a distance that the slider has been advanced, which can correspond or be associated with a degree or amount that the biasing mechanism 136 has been expanded. For example, the markings can indicate when the biasing mechanism 136 has been expanded to having a diameter of about 5 millimeters (mm), about 10 mm, about 15 mm, about 20 mm, about 25 mm, about 30 mm, about 35 mm, about 40 mm, about 45 mm, about 50 mm, about 55 mm, or about 60 mm, including all values and ranges therebetween. Various embodiments of biasing mechanism actuator 144 are described with reference to later figures below.

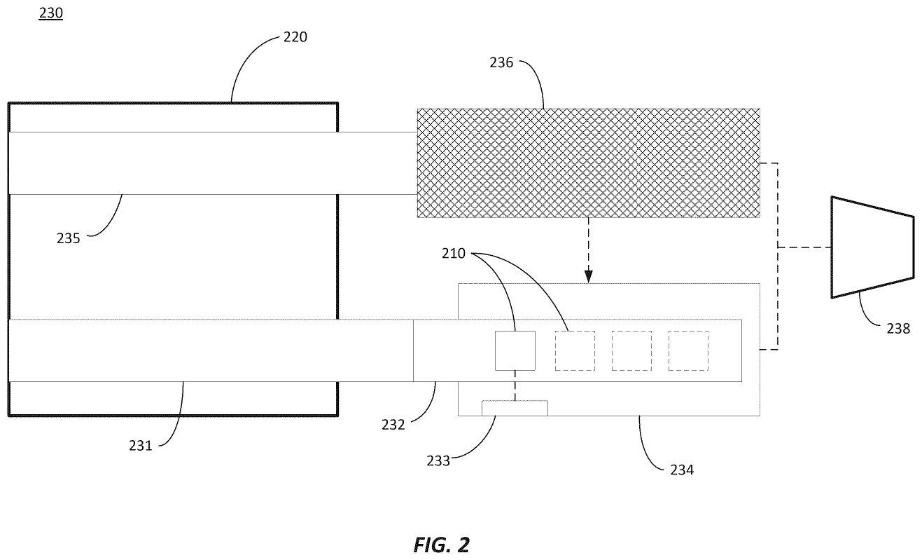

FIG. 2 shows an example of catheter assembly 230 of a suture delivery system, according to embodiments. Catheter assembly 230 can include components that are structurally and/or functionally similar to other catheter assemblies described herein, including, for example, catheter assembly 130. For example, catheter assembly 230 includes an introducer tip 238, a biasing mechanism 236, a SARE 232, a suture housing 234, a set of sutures 210 placed on SARE 232, and a deployment window 233 located in suture housing 234. Further, catheter assembly 230 includes one or more shafts 231, 235 that can be used to couple the SARE 232, the biasing mechanism 236 and/or other components of the catheter assembly 230 to actuators and/or drive components disposed at a proximal end of the suture delivery device. The catheter assembly 230 can also include an outer sheath 220 or catheter 220 that defines a lumen for receiving the shafts 231, 235 and/or other components of the catheter assembly 230.

In some embodiments, biasing mechanism 236 can include a distal end coupled to introducer tip 238, and a proximal end coupled to shaft 235. In some embodiments, shaft 235 may be directly attached to biasing mechanism 236, while in other embodiments, shaft 235 can be coupled via one or more other components to biasing mechanism 236. Similarly, in some embodiments, introducer tip 238 can be directly attached to biasing mechanism 236, while in other embodiments, introducer tip 238 can be coupled via one or more other components to biasing mechanism 236. For example, in a particular embodiment, introducer 238 can be coupled to a distal end of an additional shaft (e.g., a shaft that is disposed within a lumen of shaft 235 or an inner shaft), and the distal end of the biasing mechanism 236 can be coupled to this additional shaft. In some embodiments, when shaft 235 moves towards introducer tip 238, a distance between a distal end of shaft 235 and introducer tip 238 is reduced, thus reducing the distance between the distal and the proximal ends of biasing mechanism 236. Such movement can cause the biasing mechanism 236 to transition from an unexpanded configuration into an expanded configuration. In an example implementation, biasing mechanism 236 is an expandable mesh, e.g., a wire cage, basket, or other mesh-like structure formed of a plurality of wires that are woven or interleaved with one another. The wires can be formed of any suitable material, including, for example, nitinol, stainless steel, Elgiloy or cobalt alloy, plastic, or other materials known in the art that has the desired flexibility and strength. In some embodiments, the biasing mechanism 236 may be expanded by a different amount depending on the distance between the distal and the proximal ends of the biasing mechanism 236. Shaft 235 may be configured to move towards introducer tip 238 by at most a maximum distance, thus resulting in a maximum target expansion of biasing mechanism 236. In some embodiments, the biasing mechanism 236 in its maximum expanded state can have a diameter of about 20 mm, about 30 mm, about 40 mm, about 50 mm, about 60 mm, about 75 mm, or about 100 mm, including all values and ranges therebetween. In some cases, shaft 235 may be configured to move to a set of discrete positions (e.g., be configured to lock at a set of discrete positions), this resulting in a set of discrete expanded shapes or sizes for biasing mechanism 236. Alternatively, shaft 235 may be configured to move continuously from an initial position (e.g., corresponding to an unexpanded biasing mechanism 236) to the furthest position (i.e., move by the maximum target distance). In an example embodiment, the movement of shaft 235 is controlled by a surgeon via a slider, as further described with reference to later figures below.

In some cases, the biasing mechanism 236 may expand to a first configuration having a first shape and a first expanded volume and may expand in a second configuration having a second shape and a second expanded volume. In some embodiments, the first expanded volume may be larger than a second expanded volume. In some embodiments, the first shape may be different than the second shape. In an example embodiment, as further shown below, at least one of the first or the second shape may not be symmetric about a central axis of the biasing mechanism 236. In some embodiments, the biasing mechanism 236 may expand into an asymmetric structure or shape, e.g., in which a side of the biasing mechanism 236 that faces away from the housing 234 is configured to expand outwards (e.g., away from the housing 236) while a side of the biasing mechanism 236 that faces the housing 234 is configured to remain straight or unexpanded. In such embodiments, the side of the biasing mechanism 236 that faces the housing 234 may be generally flush and/or extend parallel to the housing 234 such that the biasing mechanism 236 when expanded can press the housing 234 against a portion of graft material and/or tissue without significantly deforming (e.g., curving or bending) the housing 234. Alternatively, in some embodiments, the biasing mechanism 236 can be configured to expand symmetrically about its central axis.

In some embodiments, shaft 235 may not be coupled to the biasing mechanism 236 but can be implemented as a sheath that covers the biasing mechanism 236. The sheath can then be retracted or advanced to allow the biasing mechanism 236 to expand out of the sheath or retract back into the sheath, respectively. In some embodiments, biasing mechanism 236 may be coupled to an elongate element (e.g., a shaft or rod) placed inside shaft 235, and shaft 235 can be configured to move relative to this elongate element. In such embodiments, biasing mechanism 236 may be an expandable mesh configured to self-expand (e.g., due to being formed from shape-memory or superelastic material) when shaft 235 is moved in a direction away from a distal end of catheter assembly 230 (i.e., in a direction away from introducer tip 238). In some cases, depending on a position of shaft 235 relative to biasing mechanism 236, biasing mechanism 236 may be fully expanded (e.g., when shaft 235 is fully retracted or moved maximally away from the distal end of catheter assembly 230), fully contracted (e.g., when shaft 235 is fully deployed or moved maximally towards the distal end of catheter assembly 230) or partially expanded when BAS 235 is partially retracted (or, in other words, partially deployed). In an example implementation of biasing mechanism 236 being an expandable mesh, the mesh may be formed from any suitable shape-memory material, such as nitinol, stainless steel, and the like.

It can be appreciated that biasing mechanism 236 formed as an expanded mesh is one possible illustrative way for implementing biasing mechanism 236, and various other implementations may be used. For example, biasing mechanism 236 may be an expandable balloon that may be inflated/deflated using any suitable fluid (e.g., gas or liquid, such as saline).

FIG. 2 further shows suture housing 234, which includes deployment window or opening 233. When biasing mechanism 236 is expanded, biasing mechanism 236 is configured to press a portion of the housing 234 against a portion of the graft and/or tissue. In particular, the biasing mechanism 236 when expanded can be configured to cause the deployment window 233 of the housing 234 to be pressed against the graft and/or tissue. The deployment window 233 can be through which one or more sutures 210 are deployed from within the housing 234. The sutures can be advanced one at a time out of the window 233 of the housing 234. Each time a suture is deployed, the biasing mechanism 236 can be undeployed or unexpanded, and the catheter assembly 230 moved such that the housing 234 is located at a different region of the graft and/or tissue. The biasing mechanism 236 can then be expanded or deployed again to press the housing 234 against the new region of graft and/or tissue. In some embodiments, the housing 234 can be configured to house or contain at least one suture, at least two sutures, at least three sutures, at least 4 sutures, at least 5 sutures, at least 6 sutures, at least 7 sutures, at least 8 sutures, at least 9 sutures, or at least 10 sutures, including all values and ranges therebetween. In an example embodiments, the housing 234 can be configured to house or contain between one and 4 sutures.

In the example embodiment shown in FIG. 2, SARE 232 can be disposed within housing 234. SARE 232 can have a proximal end that is attached to shaft 231. In use, movement of shaft 231 can cause a movement of SARE 232, such that SARE 232 can be controlled to deploy and/or retract a suture 210. In some embodiments, sutures 210 can be disposed in a distal portion of housing 234 that is distal to the window 233 such that each suture 210 can be pulled to deploy the suture through the deployment window 233. In such embodiments, the shaft 231 and SARE 232 can be moved proximally (i.e., away from introducer tip 238) to deploy a suture 210 and can be moved distally (i.e., toward introducer tip 238) to retract a suture 210 back into the housing 234. Alternatively, in other embodiments, SARE 232 may be moved towards introducer tip 238 to deploy a suture 210 and/or moved away from introducer tip 238 to retract a suture 210.

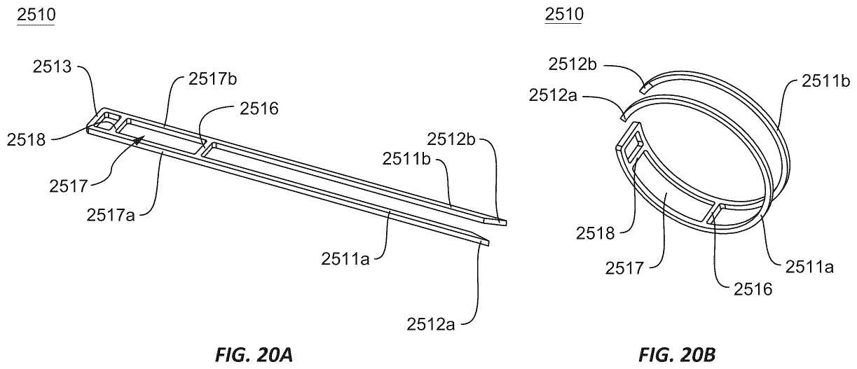

Consistent with the disclosed embodiment shown in FIG. 2, SARE 232 can move within suture housing 234. Sutures 210 can be held in a flattened configuration, as described with reference to FIG. 1, and can be received in formations disposed on a surface of SARE 232, as further described below. SARE 232 and the housing 234 collectively can constrain the sutures 210 in their flattened configuration. Sutures 210, when stored in suture housing 234, can experience elastic stress. When SARE 232 moves such that a suture (or at least a part of the suture) aligns with deployment window 233 and is deployed through the deployment window 233, the suture may curve (e.g., bend) automatically as it exits the deployment window. Example embodiments illustrating a process of a suture release are further described with reference to FIGS. 20A-20F below.

Catheter assembly 230 further includes an outer sheath 220 configured to enclose or receive at least some parts of elements 231-236, e.g., to provide a smooth profile for insertion and/or navigation of the catheter assembly 230. In some embodiments, outer sheath 220 can be configured to move relative to a distal portion of the catheter assembly 230, e.g., to cover one or more components at the distal end of the catheter assembly 230 (e.g., suture housing, biasing mechanism, SARE, etc.). As such, outer sheath 220 can be a retractable sheath that can cover the SARE, biasing mechanism, and other distal components during initial delivery of the catheter assembly 230 into the body and can be retracted prior to actuation of the biasing mechanism and SARE. In some embodiments, shafts 231, 235 can be disposed concentrically within the sheath 220. For example, shaft 235 can be disposed within a lumen of shaft 231, or vice versa.

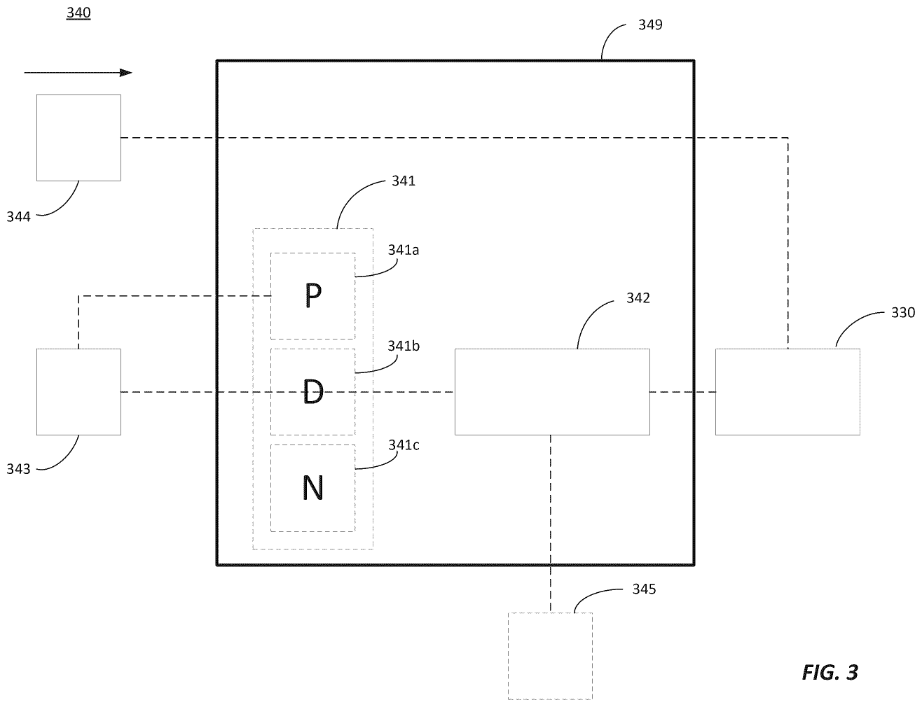

In use, sutures 210 may be configured to be deployed after biasing mechanism 236 is expanded. The biasing mechanism 236, when expanded, can anchor or hold the housing 234 and other components of the catheter assembly 230 in place within a graft and/or vessel. The anchoring provided by the biasing mechanism 236 can ensure that the sutures 210 are deployed at a precise location that does not change as the SARE 232 and/or other components of the suture delivery system are actuated. In some embodiments, sutures 210 may be prevented from being deployed until biasing mechanism 236 has been properly expanded. For example, a locking mechanism can be used to prevent deployment of sutures 210 until the biasing mechanism 236 has been deployed. Such locking mechanism can be implemented via a mode selector, as further described with reference to FIG. 3. In some embodiments, the locking mechanism can be an inter-lock that can be configured to release the suture deployment control mechanism in response to the biasing mechanism 236 being deployed, e.g., in response to the shaft 235 advancing at least a minimum or pre-set distance to at least partially deploy the biasing mechanism.

FIG. 3 shows an example of a handle assembly 340 of a suture delivery system, according to embodiments. Handle assembly 340 can include components that are structurally and/or functionally similar to other handle assemblies described herein, including for example, handle assembly 140. Handle assembly 340 can also be similar to handle assemblies described in International PCT Patent Application No. PCT/US2021/048888, incorporated above by reference. Handle assembly 340 can be coupled to a catheter assembly 330, which can be any of the catheter assemblies described herein, such as, for example, catheter assembly 130 and/or 230. Handle assembly 340 can include a handle housing 349. Handle housing 349 can house or support a suture deployment drive system 342, a suture deployment control mechanism 343, and optionally a mode selection system or mode selector 341 having configurations 341A-341C and/or a suture number selection mechanism (SNSM) 345. The handle assembly 340 can also include a biasing mechanism actuator 344.

Suture deployment control mechanism 343 and suture deployment drive system 342 allow a medical professional (e.g., a surgeon) to control deployment of a suture, e.g., through graft and/or tissue of a patient. In an example embodiment, the surgeon controls the deployment of the suture via suture deployment control mechanism 343. Suture deployment control mechanism 343 can be any suitable trigger or activation device, such as, for example, a lever, a button, a wheel, a slider, etc. When suture deployment control mechanism 343 is coupled to suture deployment drive system 342 and manipulated or actuated (e.g., depressed or pumped), suture deployment drive system 342 can cause a SARE or suture deployment element of a catheter assembly (e.g., SARE 232, which can be implemented as a sled or ribbon-shaped structure) to move to advance a suture out of a suture housing of the catheter assembly (e.g., suture housing 234). As described above, the suture deployment element can be partially disposed within the suture housing, and can interface with one or more sutures. In some embodiments, manipulation of the suture deployment control mechanism 343 can cause a first portion of the handle assembly 340 to move relative to a second portion of the handle assembly 340. The first portion of the handle assembly 340 can be coupled to the suture deployment element of the catheter assembly, while the second portion of the handle assembly 340 can be coupled to the suture housing of the catheter assembly. As such, movement of the first portion of the handle assembly 340 relative to the second portion of the handle assembly 340 causes the suture deployment element of the catheter assembly to move relative to the suture housing. In some embodiments, manipulation of the suture deployment control mechanism 343 can cause the suture deployment element to move proximally (i.e., in a direction toward the handle assembly 340) relative to the suture housing, and sutures positioned distal of a suture deployment window of the suture housing can be pulled proximally toward the suture deployment window for deployment.

In some embodiments, each individual suture can be released from the suture housing in stages. For example, with each manipulation or actuation of the suture deployment control mechanism 343 (e.g., each pump or depression of a lever, button, or other control mechanism), a suture may advance out of a suture deployment window of the suture housing by a partial amount, e.g., be partially released from deployment window 233. After a series of actuations, a suture can be fully deployed. Prior to being fully deployed, the surgeon may reverse the deployment of the suture (e.g., retract the suture). Further details of deployment drive system 342 and operations of suture deployment control mechanism 343 are discussed below.

In some embodiments, a mode selection system or mode selector 341 determines coupling of deployment drive system 342 with suture deployment control mechanism 343. Mode selection system 341 allows a user to switch between engaging suture deployment control mechanism 343 with deployment drive system 342 and disengaging suture deployment control mechanism 343 from the deployment drive system 342. In some embodiments, the suture deployment control mechanism 343 can include a drive element (e.g., a drive clasp) that can be engaged and/or disengaged from a ratchet or ratcheting tube of the deployment drive system 342 via mode selection system 341. The mode selector allows a user to switch between: a first mode illustratively identified as a “P” mode (corresponding to a first configuration 341A), which locks suture deployment control mechanism 343 in a closed configuration or position and prevents its actuation (e.g., pressing or pushing); a second mode illustratively identified as a “D” mode (corresponding to a second configuration 341B), which engages suture deployment control mechanism 343 with the deployment drive system 342 for delivering the sutures; and a third mode illustratively identified as a “N” mode (corresponding to a third configuration 341C), which disengages suture deployment control mechanism 343 from the deployment drive system 342. In the “N” mode, the suture deployment control mechanism 343 being disengaged from the drive system 342 can enable a user to retract a suture back into a suture housing, e.g., by moving a first portion of the handle assembly 340 that is coupled to a suture deployment element relative to a second portion of the handle assembly 340 that is coupled to a suture housing. In use, the mode selector 341 can be set to the “P” mode when the user desires to prevent accidental deployment of sutures, e.g., during insertion, navigation, and/or repositioning of the catheter assembly. The mode selector 341 can be set to the “D” mode when the user desires to deploy one or more sutures. And the mode selector 341 can be set to the “N” mode when the user desires to retract one or more sutures. Further embodiments of mode selection system 341 are discussed below.

Optionally, in some embodiments, the suture housing of a suture delivery device may house a few sutures (e.g., two, three, four, five, and the like). In some embodiments, the handle assembly 340 can include a SNSM 345 that is configured to index from one suture to the next during deployment of multiple sutures. For example, the handle assembly 340 can include a stepped track, whereby each step of the track corresponds to a distance traversed by the suture deployment element to fully deploy a single suture. A protrusion or tooth can be disposed within the track, and advance within the track until it contacts the end of each step of the track. This protrusion can be coupled (via one or more intervening components) to the suture deployment element. As such, once the distance for deploying one suture is traversed by the suture deployment element, the track can lock further movement of the suture deployment element to prevent accidental deployment of a second suture at the same location as a first suture. In such embodiments, a SNSM 345 can be a control mechanism that allows a user to advance the protrusion onto the next step of the track, thereby allowing a second suture to be deployed. In some embodiments, the SNSM 345 can be a wheel or knob that can be rotated to advance the protrusion into the next step of the track. The SNSM 345 can include markings that act as a counter, thereby allowing a surgeon to select the number of the suture that the surgeon is deploying. The SNSM 345 can be configured to ensure that sutures are selectively deployed one at a time. In other words, SNSM 345 can prevent two sutures from being deployed at the same location. In an example embodiment, when SNSM 345 is implemented as a wheel, a rotation of a wheel by a prescribed amount may allow selection of a suture for deployment.

Biasing mechanism actuator 344 may be any suitable slider, lever, button, and the like configured to deploy (e.g., expand) or contract biasing mechanism 236. In an example embodiment, biasing mechanism actuator 344 may be a laterally sliding element configured to move between a first position corresponding to a contracted biasing mechanism 236 and a second position corresponding to a fully expanded biasing mechanism 236. In various embodiments, positions of the laterally sliding element between the first and the second position may correspond to a partially expanded biasing mechanism 236. In some embodiments, markings or other indicia present on the handle assembly 340 can indicate to a user the degree of expansion of the biasing mechanism. For example, a first marking can indicate that the biasing mechanism is expanded to a diameter of about 5 mm if the sliding element is aligned with the first marking, and a second marking can indicate that the biasing mechanism is expanded to a diameter of about 10 mm if the sliding element is aligned with the second marking. In some embodiments, markings indicating that the biasing mechanism has been expanded to certain diameter can include, for example, one or more of 5 mm, 10 mm, 15 mm, 20 mm, 25 mm, 30 mm, 35 mm, 40 mm, 45 mm, 50 mm, 55 mm, 60 mm, etc., or any other increments and/or values therebetween.

FIG. 4 shows a schematic illustration of an embodiment of a suture 410. Suture 410 can be structurally and/or functionally similar to other sutures described herein, including, for example, sutures 110, 210, etc.

Suture 410 may have a first prong or leg 411A and a second prong or leg 411B connected via a bridging element 416 that together form a U-shaped structure. In an embodiment, the legs 411A and 411B can have proximal ends joined to each other and elongate bodies that extend parallel to one another. Both legs 411A and 411B may have sharp respective ends or sharpened tips 412A and 412B, configured to penetrate through graft and/or tissue. In some embodiments, the sharpened tips 412A, 412B can be formed or sharpened at a predetermined angle. Optionally, suture 410 may include a tail or clamping member 414 (e.g., a clamping arm), which can optionally include an engagement element 415. Tail 414 may be an elongated region. In some embodiments, tail 414 can have a length that is less than the length of the legs 411A and 411B, e.g., tail 414 can have a length of about half of the length of legs 411A and 411B. Alternatively, tail 414 can have a length that is longer than the legs 411A and 411B.

The engagement element 415 can be a laterally extending element disposed on the tail 414. The engagement element 415 can enhance the engagement (e.g., grip or traction) of the suture 410 with a SARE. The engagement element 415 can provide an additional surface, by which the SARE can push the suture 410, e.g., when deploying the suture 410 out of an opening (e.g., a deployment window). In some embodiments, the engagement element 415 may be of any suitable shape (e.g., substantially rectangular, substantially square, elliptical, oval, circular, and the like). In some embodiments, the engagement element 415 can have a width greater than a width of the rest of the tail 414. In some embodiments, the engagement element 415 can be wider than the rest of the tail 414 by a factor of about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, or about 10, inclusive of all values and ranges therebetween.

In some embodiments, tail 414 may optionally include a structure or clamp head 413 on the tail end, which may be of any suitable shape (e.g., a circular shape, for example). In an example embodiment, tail 414 may smoothly transition into structure 413 (e.g., the transition may not have sharp edges), as the absence of the sharp edges may prevent tissue damage and/or tissue rupture. Similarly, tail 414 may smoothly transition into bridging element 416 connected to the legs 411A and 411B.

In various embodiments, suture 410 is configured to curl (e.g., bend or curve) into a deployed state or configuration when released from suture housing 234. For example, suture 410 can be formed of shape-memory or superelastic material and can have a natural state that forms a curved or annular shape. In some embodiments, suture 410 can be formed from laser cutting a metallic tube. As such, in its natural state, suture 410 can form a loop or annular shape that corresponds to a cross-section of the metallic tube. In some embodiments, suture 410 can be constrained in a straightened or flattened configuration within a suture housing of a suture delivery device (e.g., suture housing 134). In the flattened configuration, the tail 414 of the suture can extend in a direction opposite to the direction of the elongate bodies of the two legs 411A and 411B. When the suture 410 is released form the suture housing, the suture 410 can revert back to its curved or natural configuration in which the tail 414 and the two legs 411 and 411B are curved and can exert forces in opposite directions such that the tail 414 and the two legs 411 and 411B are configured to collectively hold a graft and/or vessel wall together. In particular, the tail 414 and the two legs 411A and 411B can create a clamping force or a tightening effect that can maintain a section of a graft and the vessel wall relative to one another. For example, the two legs 411A can curve to form an annular shape or loop, and the tail 414 once curved can be configured to reduce an effective diameter of the loops thereby creating a tightening effect. This tightening effect or clamping can further secure the hold that the suture 410 has on the graft and/or tissue held between the legs 411A and 411B and the tail 414.

In some embodiments, a suture 410 containing legs 411A and 411B and a tail 414 may transition into a curved configuration. Tail 414 may be used to press on tissue and/or material disposed between the legs 411A and 411B to further bind or clamp together the graft and/or tissue. Tail 414 can be configured to bend as such that tail 414 has an increased bend radius past the U-shaped structure of the tails 411A and 411B, invading inside the diameter of suture 410 and improving the clamping of suture 410.

In some embodiments, a suture 410 may include regions that contain radiopaque markers, a radiopaque coating, or a nitinol alloy including a radiopaque material (e.g., a fraction of platinum or other radiopaque material for increased radiopacity). For example, tail 414, bridging element 416, or legs 411A and/or 411B may include radiopaque markers (e.g., a ring, collar, band, or plate), be coated with a radiopaque material, or include a nitinol alloy including a radiopaque material (e.g., a fraction of platinum or other radiopaque material for increased radiopacity). For example, a portion of tail 414 may include a radiopaque marker, be coated with a radiopaque material, or include a nitinol alloy including a radiopaque material (e.g., a fraction of platinum or other radiopaque material for increased radiopacity). In an example embodiment, the radiopaque material may be platinum, gold, and the like.

In various embodiments, sutures 410 can be created by laser cutting from a metal tube such as nitinol or stainless steel. In some embodiments, after laser cutting the sutures 410, the sutures 410 can be electropolished or polished via other methods to create rounded edges, e.g., to avoid unintentional cutting of tissue. Although other materials might be used, nitinol or stainless steel are exemplary examples of materials with spring-like resilience and strength, such that they can formed in the curved shape, flattened for delivery, and revert to their curved shape when fully deployed. Sutures 410 when transitioned back into their curved shape can create sufficient clamping or tightening forces that ensure that material held between the legs 411A, 411B and the tail 414 of the suture 414 are maintained together, retaining such material together against physiologic forces, for example, such as blood flow in an aorta.

In some embodiments, it may be desirable to have a suture with a smaller diameter or radius of curvature. It may be desirable to have sutures that can form a small diameter when in a curved configuration (e.g., a diameter less than about 1 mm, or less than about 2 mm, or less than about 3 mm, or less than about 4 mm, or less than about 5 mm, including all ranges and values therebetween) depending on the procedure or application. For example, procedures on anatomical structures that have a high number of surrounding structures and/or tight bends near the location of deployment may require a suture with a smaller diameter than that of a suture deployed into a structure with a flat and/or unobstructed surface. Sutures with smaller sizes, however, may be challenging to fabricate and can suffer from permanent elastic deformation. For example, nitinol wire or other shape memory materials that are used to make sutures such as those described herein may exceed their elastic limit when they are forced flat depending on the radius or width of the structure (e.g., radius or width of the suture legs or wire) and the radius of bend or effective radius of curve (ER). Therefore, it can be desirable to have a suture with features or elements that prevent permanent elastic deformation.

A structure such as a straight wire can exceed its elastic limit when the ratio of the radius of the wire (rS) to the radius of its bend or effective radius of curve (ER) exceeds about 6 percent, i.e., when rS/ER is greater than about 6 percent. For example, if a nitinol wire has a radius of 0.005″, it can exceed its elastic limits if it is bent to an effective radius of about 0.085″, i.e., 0.005/0.085=0.06. As such, when a suture leg is formed from a wire having a radius of about 2.2 mm, it may exceed its elastic limit and permanently deform when it is forced flat or placed in its flat configuration. One way to make a smaller suture (or a suture that can have a smaller curve) without exceeding the elastic limit is to reduce the wire diameter or laser-cut tube wall thickness of the suture. However, this method reduces the strength of the suture and therefore may not be adequate in some applications.

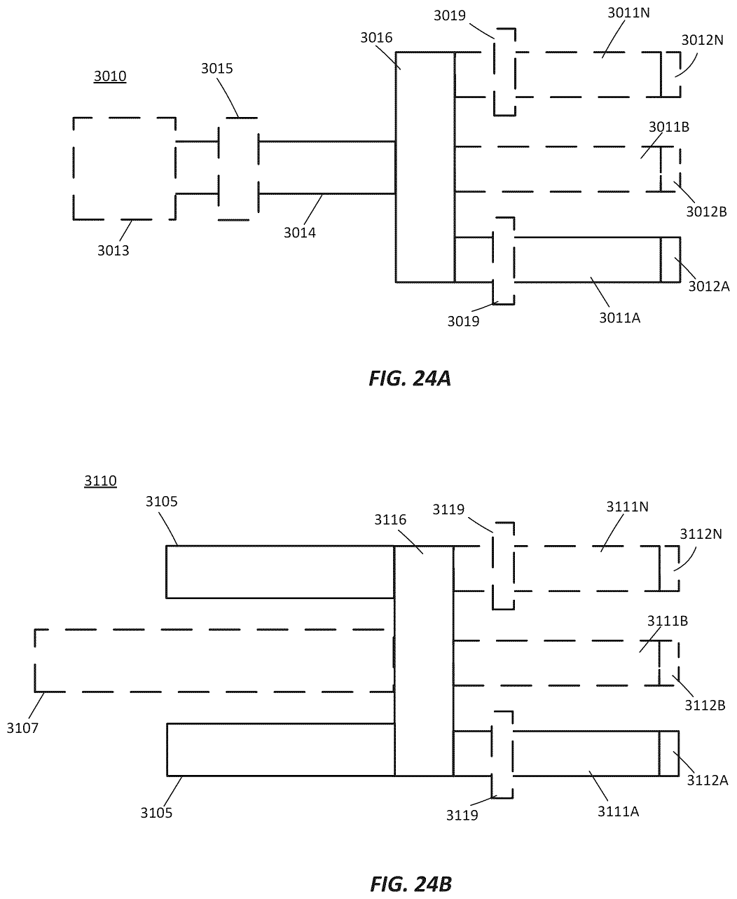

FIG. 24A shows a schematic illustration of an embodiment of a suture 3010 having curves that reduce or prevent the occurrence of permanent elastic deformation. Suture 3010 can be structurally and/or functionally similar to other sutures described herein, including, for example, sutures 110, 210, 410, etc. For example, legs 3011A through 3011N can be structurally and/or functionally similar to legs 410A and 410B. The bridging element 3016 can be structurally and/or functionally similar to the bridging element 416. The tail 3014 and clamp head 3013 may be structurally and/or functionally similar to the tail 414 and clamp head 413. As such, certain details of these elements are not described herein again with respect to FIG. 24A. In particular, the suture 3010 can be configured to transition between a flat configuration and a curved configuration, e.g., for deployment via a catheter assembly (e.g., catheter assembly 130).

Suture 3010 may include curves or bends 3019 in a direction orthogonal to its radius or bend curve. The curves 3019 can be configured to increase the effective length of the suture structure, thereby enabling the suture 3010 to have a tighter bend (or radius of curvature) in the curved configuration than a suture without curves. This can allow the suture to be smaller in its curved configuration without the downsides of having reduced strength and/or exceeding its elastic limit. Stated differently, including curves or bends 3019 in the structure of the suture 3010 in a direction orthogonal to the radius of curvature can achieve a small diameter (or radius of curvature) without exceeding the elastic limit, while also preserving the mechanical strength of the suture. Suture 3010 may include curves 3019 in the legs 3011A-N and/or the bridging element 3016. Suture 3010 may include curves 3019 in one or two dimensions. In some embodiments, suture 3010 may include curves in the legs 3011 or bridging element 3016 that form an “S” shape. While described as an “S” shape herein, the curves 3019 may have any suitable shape such that the structure can include curves in one or two directions orthogonal to the radius of curvature. For example, the curves 3019 can be a “S” shape, a spiral, loop, “U” shape, circle, or any other suitable shape. The suture 3010 formed with curves 3019 in two dimensions can have a greater effective length, thereby enabling a tighter radius of curvature compared to a suture 3010 with a smaller effective length (e.g., a suture with no curve, or a suture with a curve in one dimension). The “S” shape curve included in the legs 3011A-N of the suture 3010 may also provide the benefit of an effective stop for suture penetration.

In some embodiments, the legs 3011A-N of the suture 3010 may include a bend or curve 3019 that curves inward towards the centerline of the suture 3010. In some embodiments, the bend or curve 3019 may be shallow or small, e.g., have a displacement toward the centerline of the suture 3010 of less than about 5-20% of a lateral width of the suture in its flattened configuration, including all ranges or values therebetween. The slight bend or curve of the legs 3011A-N inward may provide higher or improved retention of the suture in the tissue after implantation. Improved retention may be useful when implanting the suture 3010 into tissue in the aorta during an endograft. In some embodiments, the suture can be cut from a tube (e.g., a nitinol tube) having a wall width of about 0.27 mm and a diameter of less than about 4.4 mm. In such embodiments, when the suture is flattened, the suture is configured to not exceed its elastic limits and therefore can revert to its curved configuration, e.g., when released from a suture housing, as described herein. In some embodiments, the width of the tube wall can be about 0.1 mm, about 0.2 mm, about 0.3 mm, about 0.4 mm, including all values and ranges therebetween, and be configured to not exceed its elastic limit when flattened. In some embodiments, the diameter of the tube can be about 0.5 mm, about 1 mm, about 1.5 mm, about 2.0 mm, about 2.5 mm, about 3.0 mm, about 3.5 mm, about 4.0 mm, about 4.5 mm, about 5.0 mm, about 5.5 mm, or about 6.0 mm, including all values and ranges therebetween, and be configured to not exceed its elastic limit when flattened.

Optionally, suture 3010 may include additional legs 301 IN in addition to the first prong leg 3011A and the second prong leg 3011B. In some embodiments, suture 3010 may have 1 leg, 2 legs, 3 legs, 4 legs, 5 legs, 6 legs, 7 legs, 8 legs, 9 legs, 10 legs, or any other suitable number of total prongs or legs 3011. Each additional leg 3011N can increase holding power or retention of the suture 3010 in comparison to a suture with fewer legs (e.g., one or two legs). A suture with additional legs 3011N may be easily deployed by the same catheter assembly (e.g., any of the catheter assemblies described herein, including catheter assembly 130) as a suture with two legs. Enhanced holding power or retention of the suture 3010 is desirable when deploying sutures in complex anatomical structures with difficult geometry, significant curving, high flow rates, surrounding anatomic structures that should be avoided, and/or fewer landing sites for sutures such as in a thoracic endograft. When deploying sutures in complex anatomical structures, it may be desirable to have a suture with a small diameter that has high holding power or retention. Adding additional prongs or legs 3011 to a suture can provide the benefit of maintaining a small diameter of the suture 3010 (or radius of curvature) while increasing holding power or retention.

In some embodiments, tail or clamp arm 3014 may optionally include a structure or clamp head 3013 on the tail end. Sutures with a clamp arm may be subject to interference by other medical devices, wires, etc. Possible causes of increased risk of interference may be significant if sutures are implanted improperly, if sutures are not properly deployed or deployed in areas of high calcium, angulation, or with other devices, where the tail (or a portion thereof) is not seated against a graft. Where the tail is not fully seated against a graft, an external device such as a guidewire may move under the clamp arm and disturb and/or dislodge the suture. As such, in some embodiments, the clamp arm 3014 may have features that prevent such interference. In some embodiments, the clamp head 3013 can be designed to have a shape (e.g., a triangle, parallelogram, square, oval, circle, trapezoid, rhombus) that is operable to deflect exterior devices away from the tip of the clamp head 3013. In one embodiment, the clamp head may have a shape with shallow angles or edges, e.g., to allow other devices to be easily deflected without getting caught in the suture. The angles of the clamp head may have smooth or gradual transitions to guide an external device away from the clamp head 3013 without disturbing the suture 3010. In some embodiments, the clamp head may have an arrowhead shape, such that an external device that comes close to the clamp head would be guided to the top of the clamp head and not below the clamp head, while allowing the external device to transition away from the suture without disturbing the suture. The shape of the clamp head (e.g., arrowhead, kite, etc.) can have edges that narrow or taper toward a proximal end of the suture, e.g., to guide external devices to that end of the suture.

While a single tail or clamp arm is depicted in FIG. 24A, it can be appreciated that a suture can have more than one tail or clamp arm, such as schematically depicted in FIG. 24B. FIG. 24B shows a schematic illustration of an embodiment of a suture 3110 having two clamp arms 3105 and optionally a shield 3107. Suture 3110 can be structurally and/or functionally similar to other sutures described herein, including, for example, sutures 110, 210, 410, 3010 etc. As such, certain details of these elements are not described herein again with respect to FIG. 24A.

Clamp arms 3105 may be elongated regions that extend from the bridging element 3116 in the direction opposite to the prong legs 3111A-N. Clamp arms 3105 can be structurally and/or functionally similar to the suture tails described herein, including, for example, tails 114, 214, 414, 3014. As such, certain details of these elements are not described herein again with respect to FIG. 24B. In some embodiments, clamp arms 3105 may extend parallel to one another. In other embodiments, clamp arms 3105 may extend angled relative to one another and/or have different portions that extend in parallel and/or are angled relative to one another. Clamp arms 3105 may be used to press on tissue and/or material disposed near or adjacent to the legs 3111A and 3111N to further bind or clamp together the graft and/or tissue.

In some embodiments, the suture 3110 may include a protective shield 3107. The protective shield 3107 may be disposed in a channel or space between the two clamp arms 3105. The protective shield 3107 may be a flexible, elongated section that is operable to protect the clamp arms 3105 from interference from an external device. The protective shield may protect the clamp arms 3105 by deflecting an external device away from the clamp arms 3105, e.g., to prevent the external device from moving underneath the clamp arms, thereby reducing disturbance to the suture 3110 and risk of the suture 3310 dislodging from the tissue. The protective shield may be more flexible than the body of the suture such that the shield may flex or bend away from an external device if the external device moves under the tip of the shield itself, thereby reducing risk of disturbing the suture 3110. The protective shield may be formed of shape-memory or superelastic material. In some embodiments, the protective shield is cut from a tube, such as, for example, a nitinol tube. As such, in some embodiments, the suture may be a unitary or monolithic structure that is cut from a single tube. In some embodiments, the protective shield includes a first “S” shape curve extending away from the bridging element 3116 and connecting to a protective tab, the protective tab connecting to a long tail extending beyond the two clamp arms 3105. The long tail may form a second “S” shape curve. The “S” shape curves provide additional flexibility to the protective shield. The first “S” shape curve may have a width that is less than the width of the prong legs 3111A-N or clamp arms 3105. For example, the first S-shaped curve may have width that is about 20% less wide than the width between the outer prong legs 3111A and 3111N. The S-shaped curve may have a width that is about 5% less wide, about 10% less wide, about 20% less wide, about 30% less wide, about 40% less wide, about 50% less wide, about 60% less wide, about 70% less wide, or about 80% less wide than the width between the outer legs, including all values and ranges therebetween. Prior to loading the suture 3110 into a catheter assembly for delivery into patient anatomy, the first S-shape can be pulled to yield (i.e., pulled beyond its yield point) such that the protective tab, after the suture is deployed and in its curved configuration, is configured to sit on top of the clamp arms 3105 and cover the channel between the clamp arms. When loaded in a delivery system, the protective tab can be pushed back into the space as it was before yielding, but be configured to pop out and shield the clamp arms upon deployment.

Similar to the suture 3010 described with reference to FIG. 24A, the suture 3110 can have one or more legs 3111A, 3111B, . . . 3111N, which can each have a sharpened tip 3112A, 3112B, . . . 3112N. The legs 3111A, 3111B, . . . 3111N can be coupled to one another via a bridging element 3116. In some embodiments, the bridging element 3116 and/or one or more legs 3111A, 3111B, . . . 3111N can also include one or more curves 3119.

FIGS. 5A-5B schematically depict a suture delivery device including a catheter assembly 530 and a handle assembly 540. FIG. 5A depicts the suture delivery device in a first configuration where a biasing mechanism 536 of the suture delivery device is in an unexpanded or undeployed configuration, while FIG. 5B depicts the suture delivery device in a second configuration where the biasing mechanism 536 is in an expanded or deployed configuration. The suture delivery device depicted in FIGS. 5A and 5B, as well as the catheter assembly 530 and the handle assembly 540, can be structurally and/or functionally similar to other suture delivery devices, catheter assemblies, and/or handle assemblies described herein. For example, catheter assembly 530 includes an introducer tip 538, a biasing mechanism 536, a suture advancement/retraction element (SARE) 532, a suture housing 534, a set of sutures 510, and a deployment window 533. Further, catheter assembly 530 includes shafts 531, 535, 537 and an outer sheath 520.

The shafts 531, 535, 537 can be concentrically positioned within the sheath 520. For example, shaft 531 can be positioned around shaft 535, which in turn can be positioned around shaft 537. As such, the shafts 531, 535, 537 and the sheath 520 can have a common longitudinal axis. Each of the shafts 531, 535, 537 can be coupled to different components of the catheter assembly 530 such that movement of the shafts 531, 535, 537 relative to one another can move and/or manipulate such components. The shaft 537 can extend through an entire length of the suture delivery device, e.g., from the introducer tip 538 to a proximal side 541 of handle assembly 540. In some embodiments, the shaft 537 can be viewed as a central shaft which is aligned with a longitudinal axis of the catheter assembly 530. The shaft 537 can define a lumen, e.g., for receiving a guidewire. As such, the suture delivery device can be configured to be advanced or guided along a guidewire that has been positioned within the patient vasculature. In some embodiments, the shaft 537 can be coupled to a distal end of the biasing mechanism 536, and the shaft 535 can be coupled to a proximal end of the biasing mechanism 536. Accordingly, the biasing mechanism can be expanded by moving shaft 535 relative to shaft 537. In some embodiments, the shaft 531 can be coupled to a proximal end of the SARE 532. The SARE 532 can have a distal portion that is disposed within the suture housing 534 and can translate or slide within the suture housing 534. As described above with reference to FIGS. 1-4 and therefore not restated in detail herein, the SARE 532 can include formations that are configured to receive sutures 510, and collectively with the suture housing 534, the SARE 532 can constrain the sutures 510 in a flattened configuration within the suture housing 534. Sutures 510 can be structurally and/or functionally similar to other sutures described herein, including, for example, suture 410.

In various embodiments, a distal portion of the catheter assembly 530, e.g., a portion including introducer tip 538, suture housing 534, and biasing mechanism 536, is configured to be fixed in place by using biasing mechanism 536 expanded within a cavity or body lumen. As described above, biasing mechanism 536 can be expanded in response to a motion of shaft 535 relative to shaft 537. In an example embodiment, biasing mechanism 536 is an expandable and contractable mesh, cage, or basket. The mesh, cage, or basket may be made from wires formed of any suitable material (e.g., nitinol, stainless steel, plastic, and the like).

Biasing mechanism 536 may be expanded when shaft 535 is moved towards introducer tip 538. In some embodiments, biasing mechanism 536 can be configured to expand asymmetrically, as schematically depicted in FIG. 5B. For example, biasing mechanism 536 can be configured to expand on a side 536a facing away from the suture housing 534 a greater amount than on a side 536b facing toward the suture housing 534. The biasing mechanism 536 can be disposed symmetrically about the shaft 537 (or substantially symmetrically about the shaft 537) in an unexpanded state, and can be disposed asymmetrically about the shaft 537 in the expanded state.

After expansion, biasing mechanism 536 can be configured to anchor in place introducer tip 538 and suture housing 534 (see FIG. 5B). For example, the biasing mechanism 536 can be configured to contact and press against the wall of a vessel to anchor itself and other components attached to it in place. In particular, side 536a of the biasing mechanism 536 can be configured to directly contact and press against the wall of a vessel and/or graft material, and side 536b of the biasing mechanism 536 can be configured to press the suture housing 534 against the wall of the vessel and/or graft material. As such, collectively, the sides of the catheter assembly 530 are pressed against the walls of the vessel and/or graft material to anchor the distal portion of the 30xample30r assembly 530 in place.

In various embodiments, other elements of catheter assembly 530 are configured to move relative to introducer tip 538 and suture housing 534. For example, shaft 531 can be configured to move in a direction away from introducer tip 538 and pull along an attached SARE 532 containing sutures 510 placed over a top surface of SARE 532. SARE 532 can be disposed within the suture housing 534 without being fixed to any portion of the housing 534. As such, SARE 532 is configured to move within housing 534 to move sutures 510. In some embodiments, sutures 510 can be constrained between formations on a top surface of SARE 532 and an internal surface 534A of housing 534 that faces the formations. Such constraining of sutures 510 results in sutures 510 being held in a flattened state. As described above, the SARE 532 can be configured to interface with each suture 510 along its entire length and therefore prevent the suture 510 from twisting or bending in undesirable ways within suture housing 534. When the SARE 532 moves proximally, i.e., toward the handle assembly 540, the SARE 532 can move sutures 510 toward the deployment window 533. As sutures 510 pass next to deployment window 533, sutures 510 are configured to curve while being released from inside of suture housing 534 through deployment window 533. FIG. 5B schematically depicts a suture 510A being released through deployment window 533.

In various embodiments, an outer sheath 520 is used to shield at least some of the elements of catheter assembly 530, e.g., during insertion and/or advancement of the catheter assembly 530. In some embodiments, outer sheath 520 can be configured to cover suture housing 534 and biasing mechanism 536 during insertion of catheter assembly 530 into a body of a patient (e.g., into an aorta of a patient) and be retracted to uncover suture housing 534 and biasing mechanism 536 (e.g., outer sheath 520 may move away from introducer tip 538 to uncover suture housing 534 and biasing mechanism 536). In various embodiments, motions of various components of suture delivery device may be controlled by a surgeon by operating various control mechanisms of handle assembly 540, as further described below.

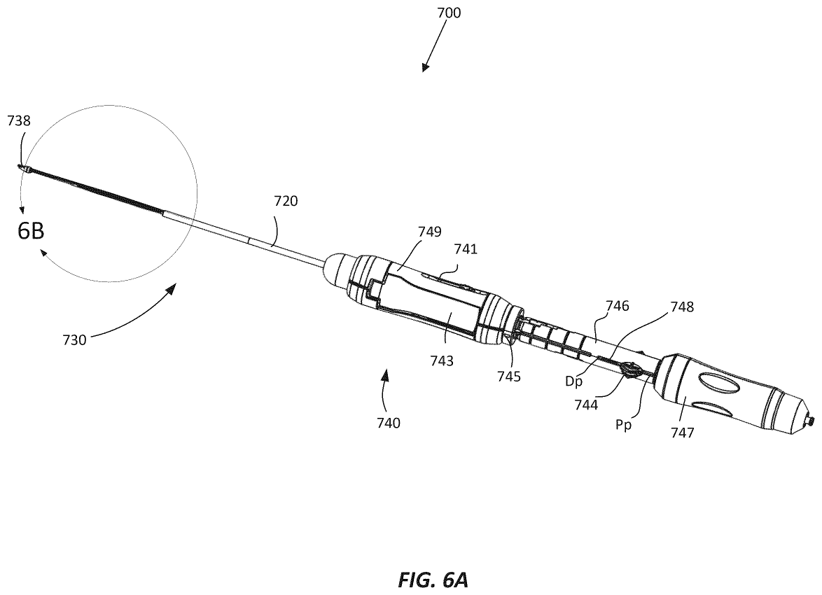

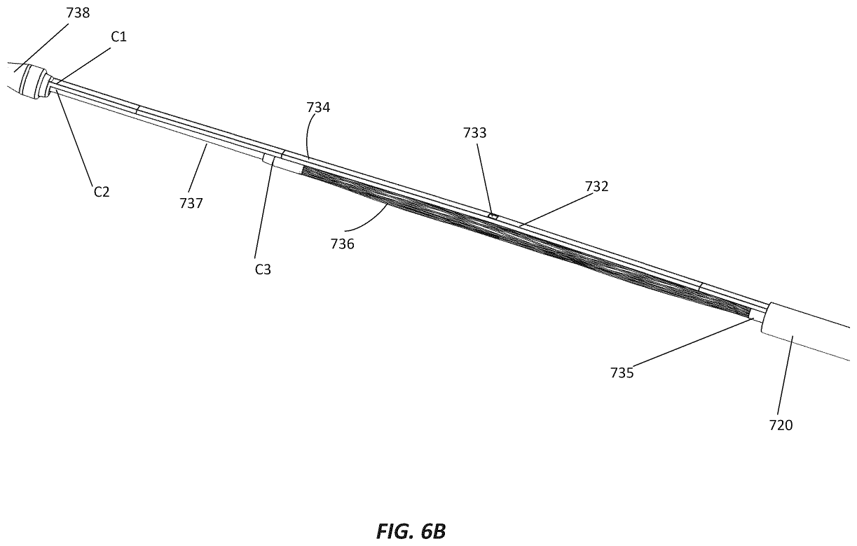

FIG. 6A shows a perspective view of an example suture delivery device 700, according to embodiments. Suture delivery device 700 can include components that are structurally and/or functionally similar to other suture delivery devices described herein, including, for example, suture delivery device 100. Device 700 includes a catheter assembly 730 and a handle assembly 740. Various details of a distal portion of catheter assembly 730 are further shown in FIG. 6B. Handle assembly 740 includes a suture deployment control mechanism 743, a mode selection system 741, a handle housing 749, an SNSM 745, a ratcheting tube 746, a biasing mechanism actuator 744, and a support handle 747.

As described above, suture deployment control mechanism 743 may interact with a drive system to facilitate deployment of sutures by catheter assembly 730. The drive system can include the ratcheting tube 746, which is coupled to the introducer tip 738 (e.g., via a central shaft). The suture deployment control mechanism 743 can be implemented as a lever that can be pumped or pressed to move the ratcheting tube 746 relative to the outer housing 749. Movement of the ratcheting tube 746 relative to the outer housing 749 can cause movement of a suture deployment element or SARE, as further described below. Mode selection system 741 controls the coupling of suture deployment control mechanism 743 and ratcheting tube 746. The ratcheting tube 746 extends within the housing 749. When mode selection system 741 is set to a first mode such as a drive or “D” mode, the suture deployment control mechanism 743 can be configured to engage with the ratcheting tube 746 such that each pump of the suture deployment control mechanism 743 causes the ratcheting tube 746 to move relative to the suture deployment control mechanism 743 and the housing 749. Alternatively, mode selection system 741 can be set to other modes, further described below, where the suture deployment control mechanism 743 is locked or not engaged with the ratcheting tube 746.

SNSM 745 is configured to configured to control selection of a suture for deployment. As described above and shown in greater detail in later figures, SNSM 745 is configured to advance a protrusion through one or more steps of a stepped channel that controls deployment of multiple sutures. Handle assembly 740 also includes a biasing mechanism actuator 744 configured to deploy (e.g., expand) a biasing mechanism of catheter assembly 730. In an example embodiment, biasing mechanism actuator 744 includes a slider that is configured to slide along a slit or channel 748 located in ratcheting tube 746. The biasing mechanism actuator 744 can be coupled to a proximal end of the biasing mechanism, e.g., via a shaft. As such, advancement of the biasing mechanism actuator 744 can be configured to move the proximal end of the biasing mechanism toward its distal end to expand it. In an example embodiment, at a distal position (Dp) within slit 748, biasing mechanism actuator 744 may be configured to fully expand a biasing mechanism of catheter assembly 730, and at a proximal position (Pp) within slit 748, biasing mechanism actuator 744 may be configured to fully contract or undeploy the biasing mechanism of catheter assembly 730. In positions between Dp and Pp, biasing mechanism actuator 744 may be configured to partially expand the biasing mechanism of catheter assembly 730.

FIG. 6B shows further details of a distal portion of catheter assembly 730. As shown, the distal portion of the catheter assembly 730 can include an introducer tip 738, a suture housing 734, portions of shafts 735, 737, and a biasing mechanism 736. The introducer tip 738 can be connected to a suture housing 734 at a connection C1 and connected to a central shaft 737 at a connection C2. Suture housing 734 can be configured to contain a SARE. Central shaft 737 is connected to a distal end of biasing mechanism 736 at a connection C3. Further, biasing mechanism 736 is connected to shaft 735 at its proximal end. Shaft 735 may include a hollow lumen within which shaft 737 is disposed. In use, shaft 735 can slide relative to central shaft 737 to deploy the biasing mechanism 736.

FIG. 6B further shows suture housing 734 with deployment window 733, through which sutures disposed within the suture housing 734 can be released. In various embodiments, suture housing 734 is configured to be flexible such that suture housing 734 can bend or bow outward (e.g., away from a longitudinal axis of the catheter assembly 730) when the biasing mechanism 736 is deployed. FIG. 6B further shows an outer sheath 720, which can surround the shafts 735, 737. In some embodiments, portions of the biasing mechanism 736, the suture housing 734, and/or other components at the distal portion of the catheter assembly 730 can be retracted within the sheath 720, e.g., during insertion or navigation of the catheter assembly 730.

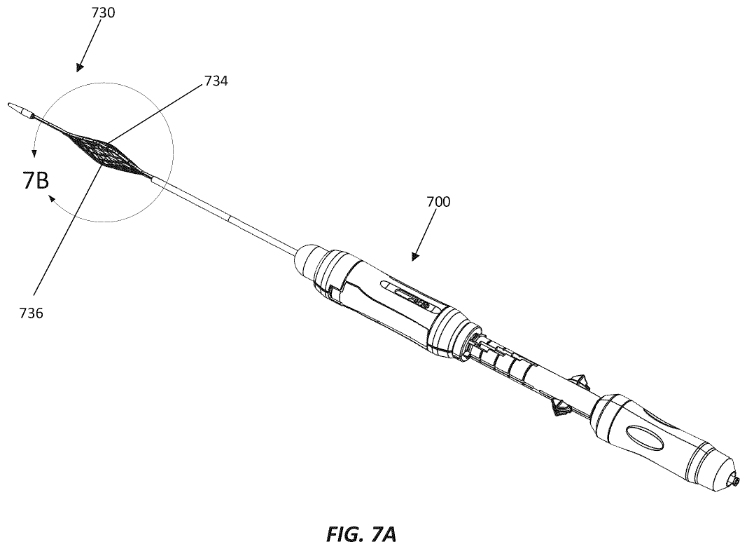

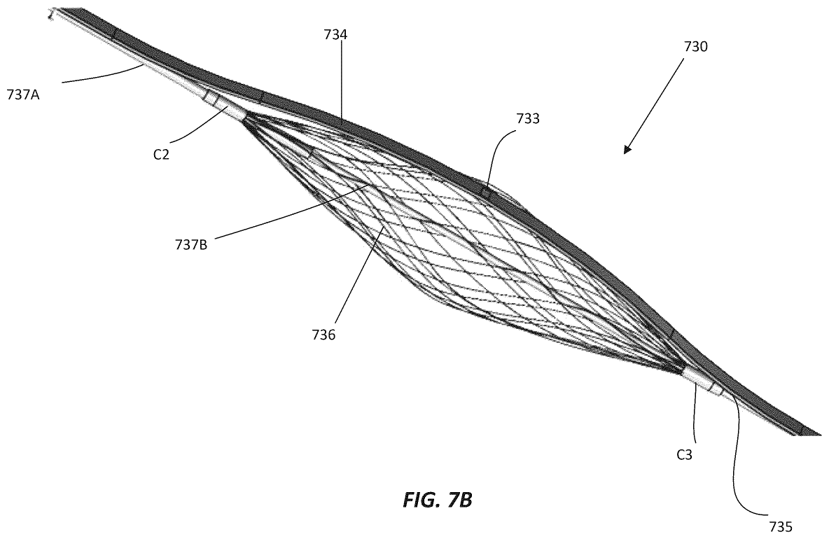

FIG. 7A shows a perspective view of suture delivery device 700 with biasing mechanism 736 expanded. Further details of a distal portion of catheter assembly 730 are shown in FIG. 7B.

As shown in FIG. 7B, a first section 737A of central shaft 737 is connected at connection C2 with biasing mechanism 736 (e.g., a distal end of biasing mechanism 736), and a second section 737B of central shaft 737 passes through biasing mechanism 736 and through a lumen of shaft 735. In an example embodiment, shaft 735 is connected to biasing mechanism 736 at a connection C3 (e.g., a proximal end of biasing mechanism 736). As shown in FIG. 7B, suture housing 734 is adjacent to biasing mechanism 736. When biasing mechanism 736 is expanded, suture housing 734 can be configured to bias or bow slightly outward, e.g., such that suture housing 734 can be pressed against a surface of a portion of graft and/or tissue. In some embodiments, the deployment window 733 can be disposed at a point along suture housing 734 that is furthest from a longitudinal axis of the catheter assembly 730 (e.g., a longitudinal axis defined by shaft 737. Such positioning of the deployment window 733 can ensure that the deployment window 733 makes contact with and presses against the portion of the graft and/or tissue, which allows a suture deployed through the deployment window 733 to penetrate through the graft and/or tissue. In an example embodiment, suture housing 734 is connected to first section 737A of shaft 737, e.g., at its distal end.

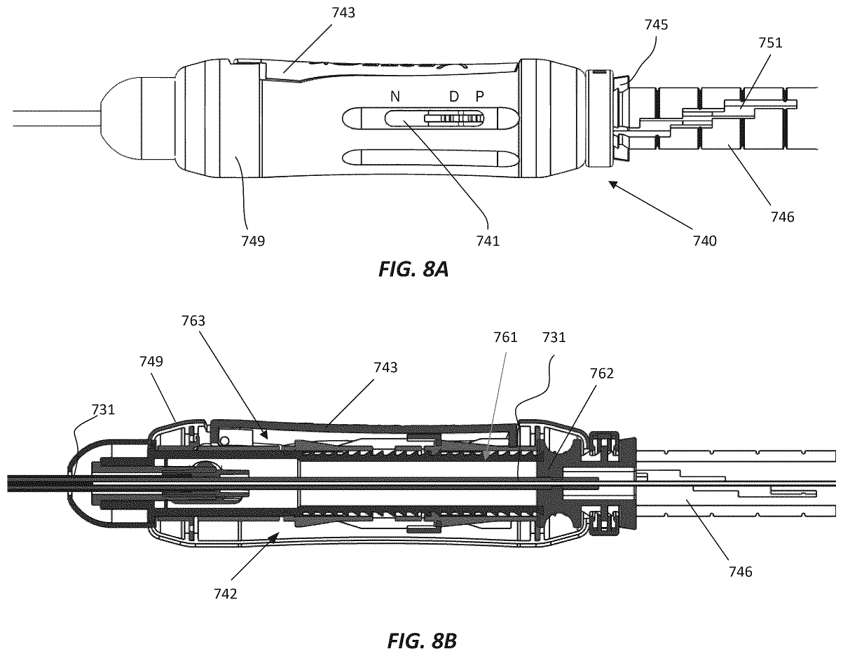

FIGS. 8A-8B illustrate further details of handle assembly 740. FIG. 8A shows a side view of a portion of handle assembly 740, including mode selection system 741, handle housing 749, suture deployment control mechanism 743, and SNSM 745. Additionally, handle assembly 740 includes ratcheting tube 746 containing a set of connected channels 751 that form a stepped channel. The ratcheting tube 746 can form a part of a suture deployment drive system 742. The suture deployment drive system 742 can also include a drive clasp 763 attached to suture deployment control mechanism 743 (e.g., lever). As suture deployment control mechanism 743 is being pressed, drive clasp 763 is configured to push against rachets or teeth 761 formed on a portion of ratcheting tube 746 disposed inside handle housing 749. While not depicted in FIGS. 8A and 8B, the ratcheting tube 746 at its proximal end is attached to the central shaft 737 of the catheter assembly 730. When the distal portion of the catheter assembly 730 is disposed at a suture site and the biasing mechanism 736 is deployed, pressing of the suture deployment control mechanism 743 causes the handle housing 749 to move proximally relative to the ratcheting tube 746, e.g., the drive clasp 763 together with handle housing 749 and suture deployment control mechanism 743 move in a direction away from introducer tip 738 and towards the proximal end of device 700. The handle housing 749 can be coupled at its proximal end to shaft 731, which in turn is coupled to a SARE that is disposed within the suture housing 734. As such, movement of the handle housing 749 relative to the ratcheting tube 746 causes the SARE to move proximally within the suture housing 734, which can move a suture toward the suture deployment window 733. In an example embodiment, each pressing of suture deployment control mechanism 743 results in a small discrete motion of drive clasp 763, handle housing 749, and suture deployment control mechanism 743 towards the proximal end of device 700. The distance traveled by drive clasp 763 due to a single pressing of suture deployment control mechanism 743 may be the same as the distance between the neighboring teeth of ratcheting tube 746. In an example embodiment, to deploy a suture via deployment window 733, suture deployment control mechanism 743 is depressed multiple times, each time advancing the suture out of the deployment window 733 a predetermined distance. After each pressing of suture deployment control mechanism 743, a spring (not shown in FIGS. 8A and 8B) may be configured to return suture deployment control mechanism 743 into an open configuration. In the open configuration, suture deployment control mechanism 743 can be positioned such that a free end of suture deployment control mechanism 743 is spaced from handle housing 749, and suture deployment control mechanism 743 can be pressed toward handle housing 749 to further move handle housing 749, suture deployment control mechanism 743, etc. proximally relative to the ratcheting tube 746. In an example embodiment, during the first pressing of suture deployment control mechanism 743, ends of legs or prongs (e.g., legs or prongs 411A and 411B of suture 410) may advance out of deployment window 733. During the second pressing of suture deployment control mechanism 743, at least a portion of the elongate bodies of the legs may advance out of the deployment window 733. During the third pressing of suture deployment control mechanism 743, the legs of suture may be entirely out of the deployment window 733, and during a fourth pressing of suture deployment control mechanism 743, a tail of a suture (e.g., tail 414) may be released from deployment window 733. It should be appreciated that having four pressing actions for releasing an entire suture is an illustrative example, and other numbers of pressing actions may be used for releasing the entire suture. In various embodiments, a partially released suture may be retracted (if the suture is not fully released) as further described below.

FIG. 8B shows that suture deployment control mechanism 743 and handle housing 749 are connected (e.g., attached) to a T-shaped element 762. In an example embodiment, T-shaped element 762 is connected to the shaft 731 of catheter assembly 730, as shown in FIG. 8B. A motion of T-shaped element 762 initiated by the motion of drive clasp 763 results in the motion of shaft 731, and as a result, the motion of a SARE attached to suture deployment element 731. The motion of SARE (e.g., towards a proximal end of device 700) results in sutures being deployed from deployment window 733 as the sutures move past deployment window 733.

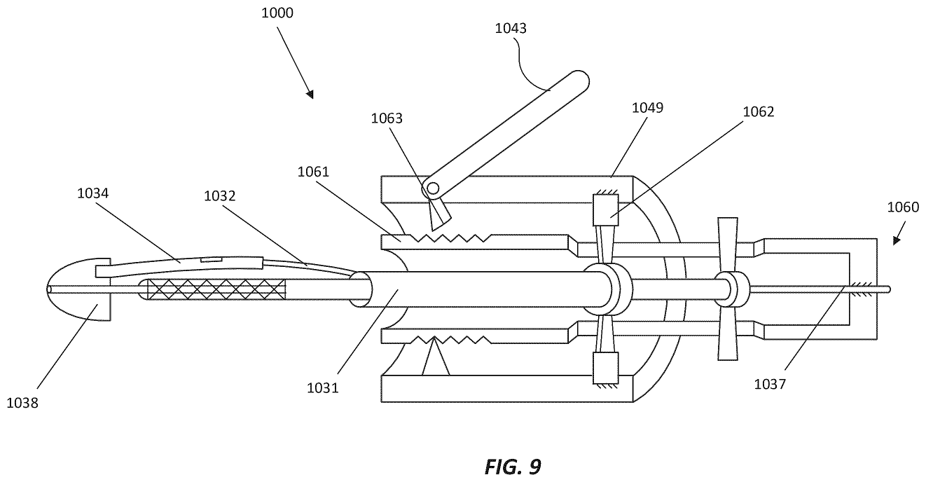

FIG. 9 illustratively depicts a suture delivery device 1000, providing a detailed view of connections for driving movement of different components of the suture delivery device 1000. Suture delivery device 1000 can include components that are structurally and/or functionally similar to components of other suture delivery devices described herein, including, for example, suture delivery devices 100 and/or 700. In an example embodiment, a suture deployment control mechanism 1043 with drive element 1063 (e.g., a drive clasp) is configured to push a ratcheting tube 1061 to move handle housing 1049, a T-shaped element 1062, a shaft 1031, and a suture advancement/retraction element 1032 proximally relative to the ratcheting tube 1061 (e.g., toward a proximal end 1060 of device 1000). The ratcheting tube 1061 in turn is coupled to a shaft 1037 that is coupled to an introducer tip 1038 of the catheter assembly. The introducer tip 1038 can be coupled to a suture housing 1034. As such, proximal movement of the handle housing 1049, the T shaped element 1062, and the shaft 1031 relative to the ratcheting tube 1061 can cause the suture advancement/retraction element 1032 to move proximally relative to the suture housing 1034, thereby advancing sutures proximally toward a deployment window of the suture housing 1034.

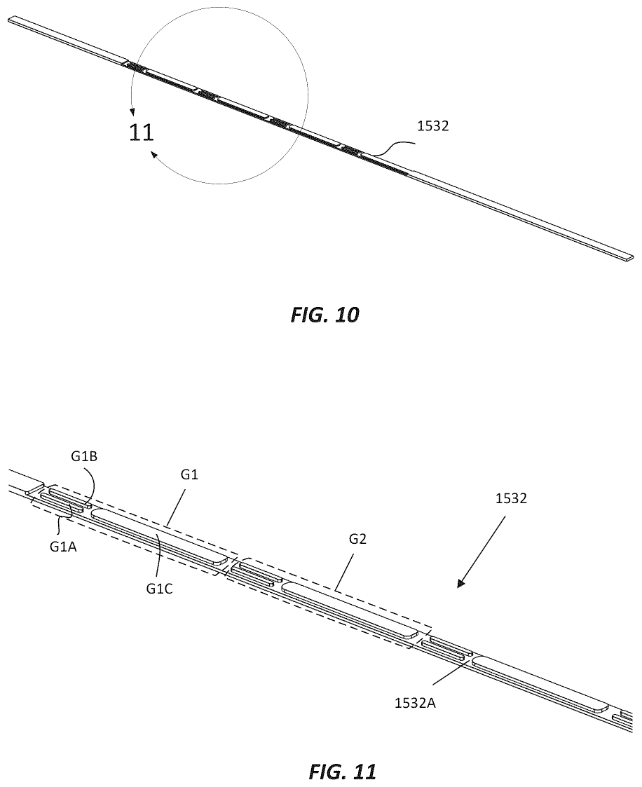

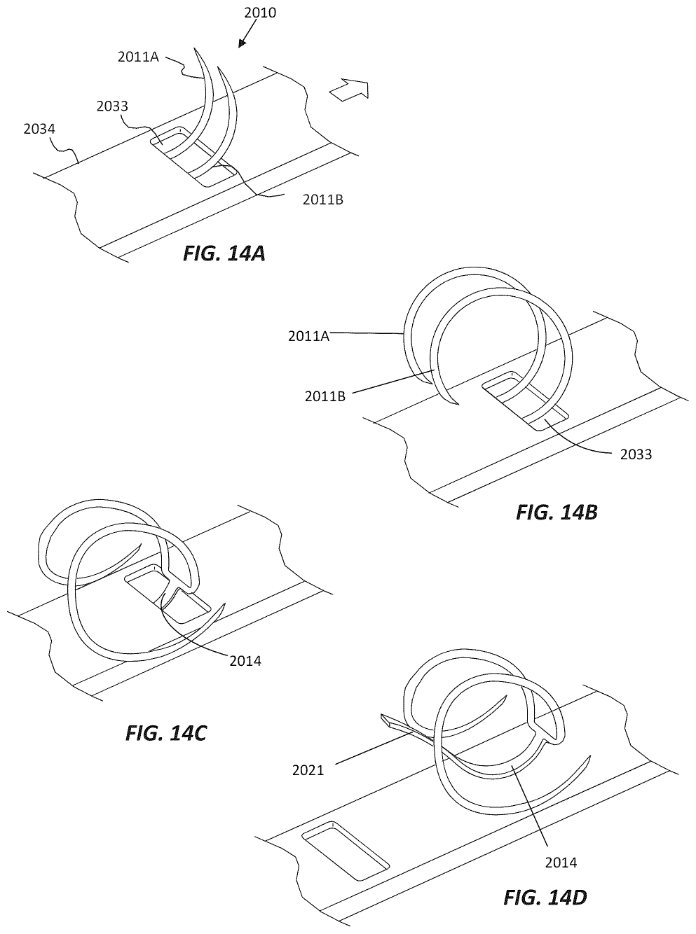

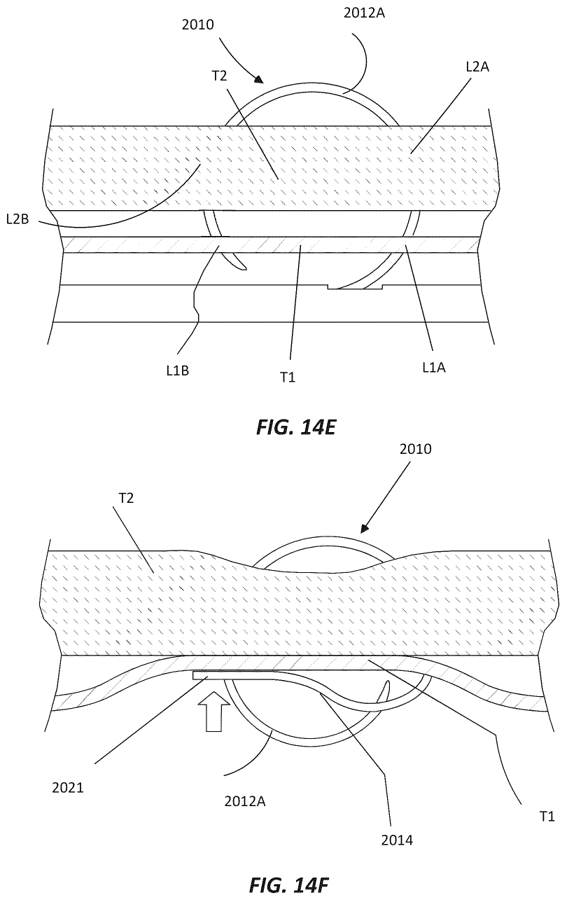

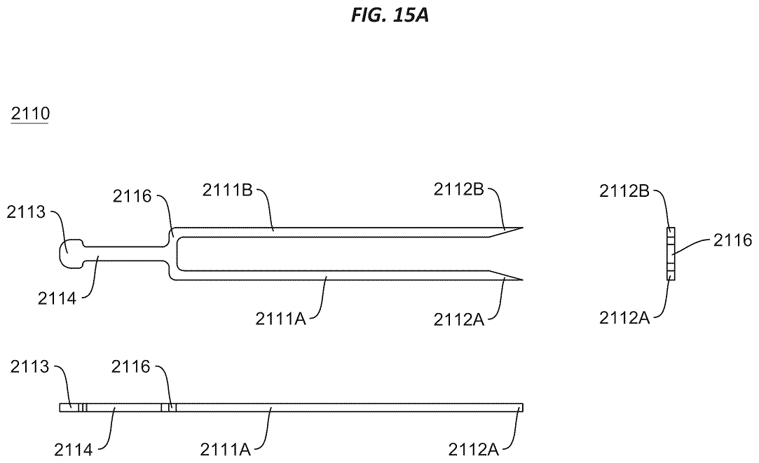

FIGS. 10-11 show various example suture advancement/retraction elements or SAREs configured to support various respective sutures shown in FIGS. 14A-14C 12A-12C. Such suture advancement/retraction elements can be functionally and/or structurally similar to other suture advancement/retraction elements described herein (e.g., suture advancement/retraction element 132, 232). Further FIG. 13, shows an embodiment of suture advancement/retraction element with an example suture placed over the element.

Consistent with disclosed embodiments, a SARE 1532 is shown in FIGS. 10 and 11. SARE 1532 is substantially an elongated rectangular prism. FIG. 11 shows that SARE 1532 includes a set of groups G1, G2, and so on, of protrusions, each group containing a first set of protrusions (e.g., G1A and G1B) and a second protrusion G1C. In an example embodiment, protrusions are placed over an indented region 1532A. In an example embodiment, top surfaces of protrusions G1A, G1B, and G1C are configured to be adjacent to or near the internal surface of the top side of a suture housing.

SARE 1532 is configured to support sutures containing tails (e.g., sutures that include a tail region, such as tail region 414, as shown in FIG. 4). An example suture may be placed over a surface of indented region 1532A, adjacent to protrusions G1A, G1B, and G1C. In various embodiments, a suture placed over the surface of indented region 1532A is flattened when SARE 1532 is located within a suture housing, as the suture may not have any room to expand and curl.

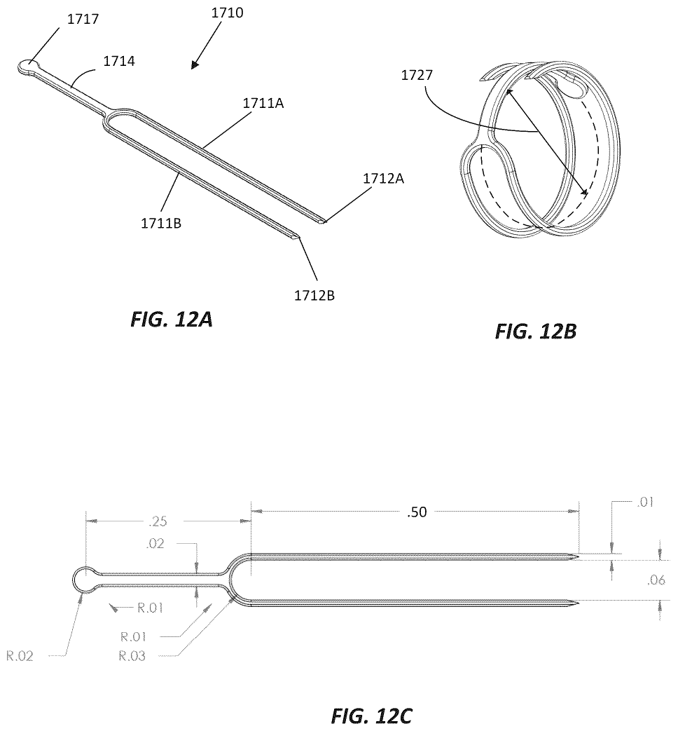

FIG. 12A shows an example embodiment of a suture containing a tail region 1714. Similar to suture 1410, suture 1710 has prongs 1711A and 1711B having respective sharp ends 1712A and 1712B configured to penetrate tissue. In an example embodiment, prongs 1711A and 1711B are connected by a connecting to tail region 1714. FIG. 12A shows a flattened view of suture 1710 and FIG. 12B shows a curled suture 1710 (e.g., suture 1710 may curl after being released from a suture housing). In an example embodiment, suture 1710 curls in a substantially circular shape, with a diameter 1727 having a value of about 0.16 inches. In an36xamplee embodiment, diameter 1727 may vary by as much as about 50% from the above value. FIG. 12C shows an example illustrative dimension for suture 1710. In an example embodiment, the length of prongs 1711A and 1711B may be in a range of about a few tenths of an inch (e.g., in a range of about 0.1 inches to about 1 inch, including all sub-ranges and values therebetween). In an example embodiment, the length of prongs 1711A and 1711B may have a value of about 0.5 inches. In various embodiments the distance between prongs 1711A and 1711B may also be in a range of about a few hundredth of an inch to a few tenth of an inch (e.g., in a range of about 0.01 inches to about 0.2 inches, including all sub-ranges and values therebetween), In an example embodiment, the distance between prongs 1711A and 1711B may have a value of about 0.06 inches.

In an example embodiment, tail region 1714 may have a length in a range of a few tenths of an inch and may be connected to prongs 1711A and 1711B via a connecting element 1716. In various embodiments, a width of prongs 1711A and 1711B may be a fraction of an inch (e.g., may be in a range of 0.01-0.05 inches). In various embodiment, the size, shape, and thickness of suture 1710 are selected based on a type of tissue that needs to be sutured. For example, for a thicker tissue, larger sutures may be selected and for a thinner, gentler tissues, smaller sutures may be selected. In various embodiments, values for the thickness of prongs 1711A and 1711B, and for the radius of curvature for connecting element 1716, may vary by as much as about 50% from the possible values for these elements, as described above.

In various embodiments, a suture advancement/retraction element is selected based on a type (e.g., size, shape, and thickness) of sutures that need to be used for the surgical procedure.

In various embodiments, suture 1710 may be laser cut from a suitable sheet or tube of a material (e.g., nitinol, plastic, stainless steel, and the like).

FIG. 13 depicts an example of SARE 1532 implemented as a sled or ribbon-like structure with suture 1710 placed over a portion of a surface of SARE 1532 (e.g., suture 1710 is placed over surface 1532A) adjacent to protrusions or formations G1A, G1B, and G1C. The formations G1A, G1B, and G1C can include two smaller formations G1A and G1B that are configured to radially surround a portion of a tail of the suture 1710 and a larger formation G1C that is disposed between legs 1711A and 1711B of suture 1710. When the SARE 1532 is placed within a suture housing, the formation G1C can radially bound an inside of the legs 1711A and 1711B, while an outside of the legs 1711A and 1711B can be bound by an inner surface of the suture housing such that the formation and the suture housing collectively constrain the legs 1711A and 1711B of the suture in a radial direction. Such radial constraining can prevent twisting of the legs 1711A and 1711B of the suture and ensure that the legs 1711A and 1711B of the suture remain parallel to one another. When the suture then exits a deployment window of the housing, the legs 1711A and 1711B of the suture can be configured to exit parallel to one another and at an angle that is substantially normal relative to a plane of the window (e.g., at an angle of between about 70 degrees and about 110 degrees, including all subranges and values, relative to the window).

FIGS. 14A-14F indicate various steps (or stages) of deployment of a suture consistent with disclosed embodiments. At a first step, as shown in FIG. 14A prongs 2012A and 12012B of suture 2010 are configured to deploy through a deployment window 2033 of a suture housing 2034. In an example embodiment, SARE 2032 is pulled in a direction as indicated by arrow 2020. FIG. 14B shows a second step, at which prongs 2012A and 2012B are substantially deployed, FIG. 14C shows a third step at which deployment of prongs 2012A and 2012B is complete, and a tail region 2014 may need to be deployed. At a fourth step, as shown in FIG. 14D, tail region 2014 is deployed. A tail end 2021 may be configured to push against a tissue (not shown in FIG. 14D) such that suture 2010 is well bound to the graft and tissues which it is suturing.

FIGS. 14E and 14F show suture 2010 penetrating and binding materials and/or tissues T1 and T2. For purposes of illustration, FIGS. 20E and 20F depict certain dimensions of suture 2010 and materials and/or tissues T1 and T2 that facilitate describing the clamping effect of suture 2010 on materials and/or tissues T1 and T2. Nevertheless, it can be appreciated that in practical applications, the dimensions of suture 2010 and materials and/or tissues T1 and T2 may be significantly different, e.g., where materials and/or tissues T1 and T2 are thicker than shown and suture 2010 is configured to be buried in materials and/or tissues T1 and T2. As such, it should be noted that the suture 2010 and materials and/or tissues T1 and T2 depicted in FIGS. 14E and 14F may not be drawn to scale. In an example embodiment, as shown in FIG. 14E, a prong (e.g., prong 2012A) of suture 2010 is configured to penetrate tissue T1 at a location L1A, penetrate tissue T2 at a location L2A, and then, due to curling of suture 2010, penetrate again tissue T2 at a location L2B, and penetrate again tissue T1 at a location L1B. Upon complete release of suture 1210, as shown in FIG. 14F, tail region 2014 is configured to press on tissue T1, using tail end 2021. By pressing with tail end 2021, suture 2010 is configured to improve binding between tissue T1 and T2.

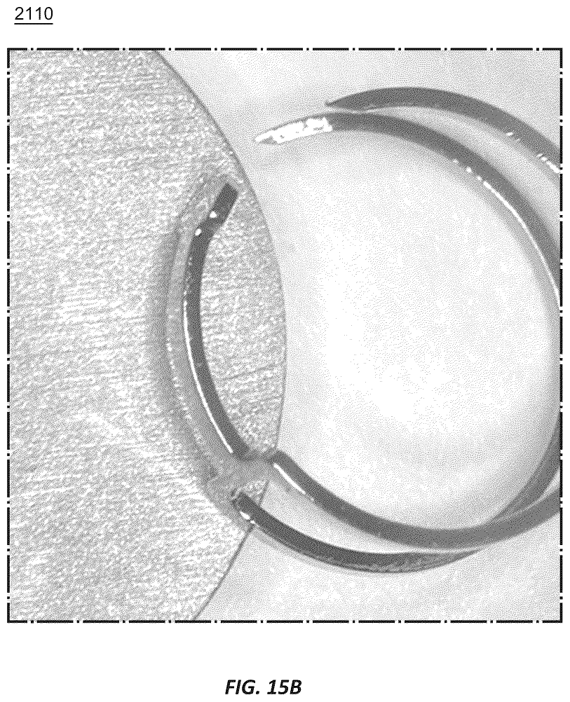

FIG. 15A shows a suture 2110 with angled tips in a flat configuration, according to an embodiment. As shown, the suture 2110 includes a first prong leg 2111A and a second prong leg 2111B connected via a bridging element 2116, a tail 2114, and a structure 2113 on the end of the tail 2114. Both legs 2111A and 2111B may have sharp respective ends or sharpened tips 2112A and 2112B, configured to penetrate through graft and/or tissue. FIG. 15A shows a plan view of the suture 2110 on the top left of the figure, a side view of the suture 2110 on the bottom left of the figure, and a front view of the suture 2110 on the top right of the figure. In some embodiments, the first prong leg 2111A, the second prong leg 2111B, the sharpened tips 2112A, 2112B, the structure 2113, the tail 2114, and the bridging element 2116 can be the same or substantially similar to the first prong leg 411A, the second prong leg 411B, the sharpened tips 412A, 412B, the structure 413, the tail 414, and the bridging element 416, as described above with reference to FIG. 4, or other sutures described herein (e.g., suture 1710). Thus, certain aspects of the first prong leg 2111A, the second prong leg 2111B, the sharpened tips 2112A, 2112B, the structure 2113, the tail 2114, and the bridging element 2116 are not described in greater detail herein.

As shown, the sharpened tips 2112A, 2112B have angled ends or surfaces with angles of 15 degrees. The angled ends can be configured to effectively penetrate through tissue, e.g., to place the suture 2110 in use. In some embodiments, the angled ends or surfaces of the sharpened tips 2112A, 2112B can have angles of at least about 5 degrees, at least about 10 degrees, at least about 15 degrees, at least about 20 degrees, at least about 25 degrees, at least about 30 degrees, at least about 35 degrees, at least about 40 degrees, at least about 45 degrees, at least about 50 degrees, at least about 55 degrees, at least about 60 degrees, at least about 65 degrees, at least about 70 degrees, at least about 75 degrees, at least about 80 degrees, or at least about 85 degrees. In some embodiments, the angled ends or surfaces of the sharpened tips 2112A, 2112B can have angles of no more than about 90 degrees, no more than about 85 degrees, no more than about 80 degrees, no more than about 75 degrees, no more than about 70 degrees, no more than about 65 degrees, no more than about 60 degrees, no more than about 55 degrees, no more than about 50 degrees, no more than about 45 degrees, no more than about 40 degrees, no more than about 35 degrees, no more than about 30 degrees, no more than about 25 degrees, no more than about 20 degrees, no more than about 15 degrees, or no more than about 10 degrees.

Combinations of the above-referenced angles of the sharpened tips 2112A, 2112B are also possible (e.g., at least about 5 degrees and no more than about 90 degrees or at least about 10 degrees and no more than about 40 degrees), inclusive of all values and ranges therebetween. In some embodiments, the angled ends or surfaces of the sharpened tips 2112A, 2112B can have angles of about 5 degrees, about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, or about 90 degrees. In some embodiments, the sharpened tip 2112A can have the same or a substantially similar angle to the sharpened tip 2112B. In some embodiments, the angled end or surface of the sharpened tip 2112A can have an angle greater than that of the sharpened tip 2112B. In some embodiments, the angled end or surface of the sharpened tip 2112A can have an angle less than that of the sharpened tip 2112B.

In some embodiments, the sharpened tips 2112A, 2112B can be rounded or slightly rounded. In some embodiments, the sharpened tips 2112A, 2112B can be electropolished to an extent of about 10%, about 15%, about 20%, about 25%, or about 30%, inclusive of all values and ranges therebetween. In some embodiments, the sharpened tips 2112A, 2112B can be laser cut. In some embodiments, the prong legs 2111A, 2111B can have a rectangular cross section. In some embodiments, the cross sections of the prong legs 2111A, 2111B can be rounded slightly during electropolishing. In some embodiments, the prong legs 2111A, 2111B can have a thickness defined by a wall thickness of a tube the suture 2110 is cut from. In some embodiments, the prong legs 2111A, 2111B can have a thickness of about 0.1 mm, about 0.15 mm, about 0.2 mm, about 0.25 mm, about 0.3 mm, about 0.35 mm, about 0.4 mm, about 0.45 mm, or about 0.5 mm, inclusive of all values and ranges therebetween. In some embodiments, the prong legs 2111A, 2111B can have a width of about 0.15 mm, about 0.2 mm, about 0.25 mm, about 0.3 mm, about 0.35 mm, about 0.4 mm, about 0.45 mm, about 0.5 mm, or about 0.55 mm, inclusive of all values and ranges therebetween. The width and thickness of the prong legs 2111A, 2111B can provide stability of twist resistance of the prong legs 2111A, 2111B, such that the prong legs 2111A, 2111B track straight as they exit the deployment housing window.

FIG. 15B shows a photograph of the suture 2110 with angled tips in a curved configuration, according to an embodiment. After being expelled from a catheter assembly, the suture 2110 takes on a curved shape as it penetrates the tissue and/or graft. As shown, the suture 2110 has curled in a substantially circular shape. Alternatively, a suture can curve in an elliptical or oval shape. In some embodiments, the diameter or a distance across the curved suture 2110 can be about 0.5 mm, about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm, inclusive of all values and ranges therebetween.

In some embodiments, the suture 2110 can be laser cut. Laser cutting the suture 2110 on a nitinol tube can allow for large variations in the length of the prong legs 2111A, 2111B, as desired. In its curved form, the suture 2110 can have large variations in leg length (e.g., up to about 360 degrees around). This can allow for some overlap of the prong legs 2111A, 2111B to a clamping arm or tail, if desired. In some cases, the prong legs 2111A, 2111B curve past about 200 degrees from entry, depending on entry angle, such that there is a hook relative to the surface of the graft and the tip of the suture leg is pointing back towards the aorta at an angle,, e.g., at least orthogonally. The suture 2110 can be sufficiently long but not so long, such that the suture 2110 is difficult to deploy, or that the sharpened tips 2112A, 2112B penetrate back through the graft. In some embodiments, the suture 2110 can have a length such that the sharpened tips 2112A, 2112B are adjacent or approximately adjacent to the proximal structure 2113 or end of the tail 2114 opposite the prong leg ends.

In some embodiments, the angle that the length of the prong legs 2111A, 2111B spans when the suture is in a curved configuration can be about 250 degrees, about 260 degrees, about 270 degrees, about 280 degrees, about 290 degrees, about 300 degrees, about 310 degrees, about 320 degrees, about 330 degrees, about 340 degrees, or about 350 degrees, inclusive of all values and ranges therebetween. For example, for a suture of 5 mm diameter, a length of about 11.8 mm is approximately 75 percent of the circumference or about 270 degrees.

In some embodiments, the bridging element 2116 can be squared or have a square planform relative to the prong legs 2111A, 2011B, rather than having a shape that is curved or forms a continuous radius with the adjacent prong legs 2111A, 2111B. A square planform can provide additional anti-twist properties to the prong legs 2111A, 2111B, as there is an abrupt termination of the prong legs 2111A, 2111B, rather than a gradual transition with a radius. A gradual radius transition can also increase the cross section of the suture at any one point in the radius, orthogonal to the central axis of the planform of the suture 2110. This larger cross section also migrates away from the central axis of the prong legs 2111A, 2111B. This change in cross section can increase the potential for the suture 2110 in its flattened form (e.g., a high energy state), to impart a twist in the prong legs 2111A, 2111B as the prong legs 2111A, 2111B exit the window. Therefore, a squared planform bridging element 2116 can reduce the potential for twisting of the prong legs 2111A, 2111B.

A clamping arm length (e.g., a length of the clamping arm or tail) can represent a distance from the inside edge of the bridging element 2116 to the end of the structure 2113. The clamping arm length can be selected to be sufficiently long to provide a clamping affect. The clamping arm can be configured to provide a resistance to motion of the suture (e.g., it prevents the suture 2110 from backing out). The clamping arm length can be sufficient to ensure complete penetration of the prong legs 2111A, 2111B, while also being short enough to be pushed out of the delivery housing without significant resistance. In some embodiments, the clamping arm length can be about 2 mm, about 2.5 mm, about 3 mm, about 3.5 mm, about 4 mm, about 4.5 mm, about 5 mm, about 5.5 mm, or about 6 mm, inclusive of all values and ranges therebetween. The clamping arm can have a suitable length that provides the suture 2110 with sufficient holding capability, allows the suture 2110 to penetrate the tissue completely, and allows the suture 2110 to be reliably pushed out of the delivery housing of a suture delivery device. Longer suture clamps can show increased difficulty in deployment and shorter suture clamp lengths may not provide an adequate clamping effect.

A suture clamp width can represent the clamp area between the bridging element 2116 and the structure 2113. The suture clamp width can be selected to enable the suture 2110 to be reliably pushed out of a deployment window without distortion, provide adequate clamping strength, and not create undue forces during deployment. The tail 2114 counters or extends in an opposite direction from the prong legs 2111A, 2111B. In some embodiments, the tail 2114 can have a width of about 0.3 mm, about 0.35 mm, about 0.4 mm, about 0.45 mm, about 0.5 mm, about 0.55 mm, or about 0.6 mm, inclusive of all values and ranges therebetween. In some cases, a larger width of the tail 2114 can cause higher frictional forces inside the housing during deployment.

The structure 2113 provides an enlarged atraumatic surface that engages with the graft material to mitigate the effect of the clamping pressure and can provide features that engage with the deployment SARE to allow for the SARE to pull the suture 2110 into the SARE easily during loading. The structure 2113 provides a surface that the SARE engages to push the suture 2110 out via the deployment window. In some embodiments, the structure 2113 can have a large planform and have a proximal radius (e.g., a curved or atraumatic surface), e.g., for benignly engaging the graft material. In some embodiments, the structure 2113 can be short in length, such that it does not encroach on the flexibility of the tail 2114. In some embodiments, the structure 2113 can have distal shoulders that adequately engage features on the SARE for loading. In some embodiments, the structure 2113 can have a width of about 0.5mm, about 0.6 mm, about 0.7 mm, about 0.8 mm, about 0.9 mm, about 1 mm, about 1.1 mm, about 1.2 mm, about 1.3 mm, about 1.4 mm, or about 1.5 mm, inclusive of all values and ranges therebetween. In some embodiments, the structure 2113 can have a width of about 0.1 mm, about 0.2 mm, about 0.3 mm, about 0.4 mm, about 0.5 mm, about 0.6 mm, about 0.7 mm, about 0.8 mm, about 0.9 mm, or about 1 mm, inclusive of all values and ranges therebetween.

FIGS. 16A-16B show manufacturing of angled or sharpened tips of sutures, according to various embodiments. Such sutures can be any of those described herein, including for example, suture 2110. As shown in FIG. 16A, the tips of the suture are advanced through a deployment window in an unsharpened state such that they are exposed. FIG. 16B shows the tips of the suture contacting a fine diamond grit grinding wheel such that the tips of the suture are at an angle of about 30 degrees and orthogonal to the laser cut angle. This procedure can provide a sharp tip with a minimal amount of grind (e.g., about 25% of the length of laser cut angle). This can be a light touch grind and can take a few seconds per tip. The sharpened tip as manufactured using this method can perform well at penetrating graft material and aorta. This grinding strategy has several advantages. It can minimize or reduce the amount of ground surface, e.g., such that there is less potential for oxidation and therefore passivation. This strategy can also ease fixturing, enhance process control, and/or repeatability. The process also has a short duration (e.g., about 2-4 seconds per grind).

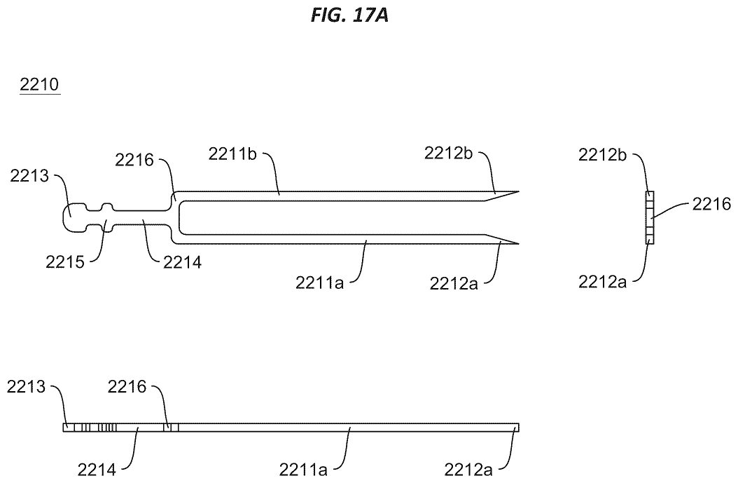

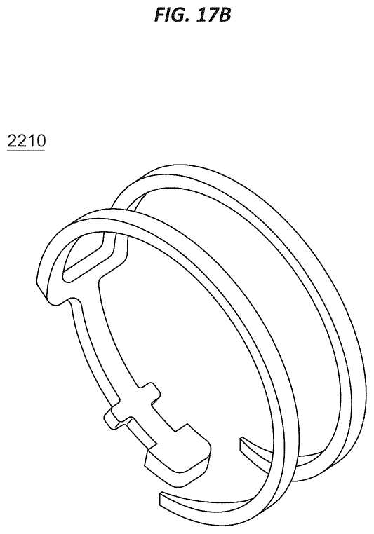

FIG. 17A shows a suture 2210 with angled tips and an engagement portion, in a flat configuration, according to an embodiment. As shown, the suture 2210 includes a first prong leg 2211A and a second prong leg 2211B connected via a bridging element 2216, a tail 2214, a structure 2213 on the end of the tail 2214, and an engagement element 2215. Both legs 2211A and 2211B may have sharp respective ends or sharpened tips 2212A and 2212B, configured to penetrate through graft and/or tissue. FIG. 17A shows a plan view of the suture 2210 on the top left of the figure, a side view of the suture 2210 on the bottom left of the figure, and a front view of the suture 2210 on the top right of the figure. In some embodiments, the first prong leg 2211A, the second prong leg 2211B, the sharpened tips 2212A, 2212B, the structure 2213, the tail 2214, and the bridging element 2216 can be the same or substantially similar to the first prong leg 2111A, the second prong leg 2111B, the sharpened tips 2112A, 2112B, the structure 2113, the tail 2114, and the bridging element 2116, as described above with reference to FIGS. 15A-15B. In some embodiments, the engagement portion 2215 can be the same or substantially similar to the engagement portion 415, as described above with reference to FIG. 4. Thus, certain aspects of the first prong leg 2211A, the second prong leg 2211B, the sharpened tips 2212A, 2212B, the structure 2213, the tail 2214, the engagement portion 2216, and the bridging element 2216 are not described in greater detail herein.

The engagement portion 2215 provides one or more additional surfaces for portions of a sled to push and advance the suture 2210, e.g., for deploying and/or retracting the suture. In some embodiments, the engagement portion 2215 can be disposed on the tail 2114 and have a length along a longitudinal axis of the tail 2114 of about 0.1 mm, about 0.2 mm, about 0.3 mm, about 0.4 mm, about 0.5 mm, about 0.6 mm, about 0.7 mm, about 0.8 mm, about 0.9 mm, or about 1 mm, inclusive of all values and ranges therebetween. In some embodiments, the engagement portion 2215 can have a width (e.g., laterally extending dimension) of about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, inclusive of all values and ranges therebetween. In some embodiments, the engagement portion 2215 can have a circular or substantially circular shape. In some embodiments, the engagement portion 2215 can have an elliptical or oval shape. In some embodiments, the engagement portion 2215 can have a linear or elongated shape, e.g., in directions that extend laterally out from the tail 2114.

FIG. 17B shows the suture 2210 in a curved configuration. In some embodiments, the curving properties of the suture 2210 can be the same or substantially similar to the curving properties of the suture 2110, as described above with reference to FIGS. 15A-15B. Thus, certain aspects of the suture 2210 and the curving of the suture 2210 are not described in greater detail herein.

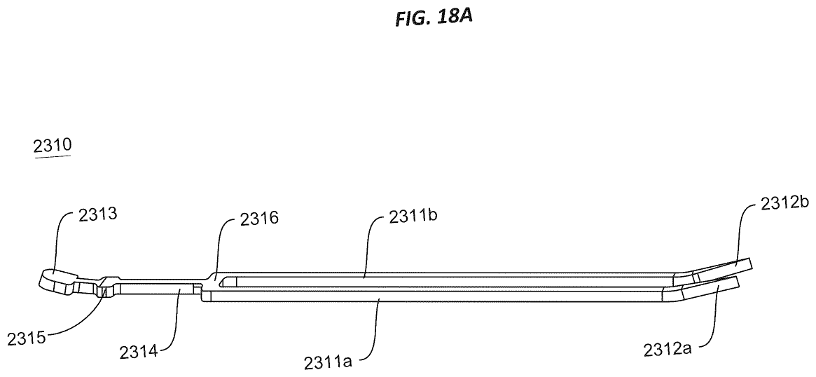

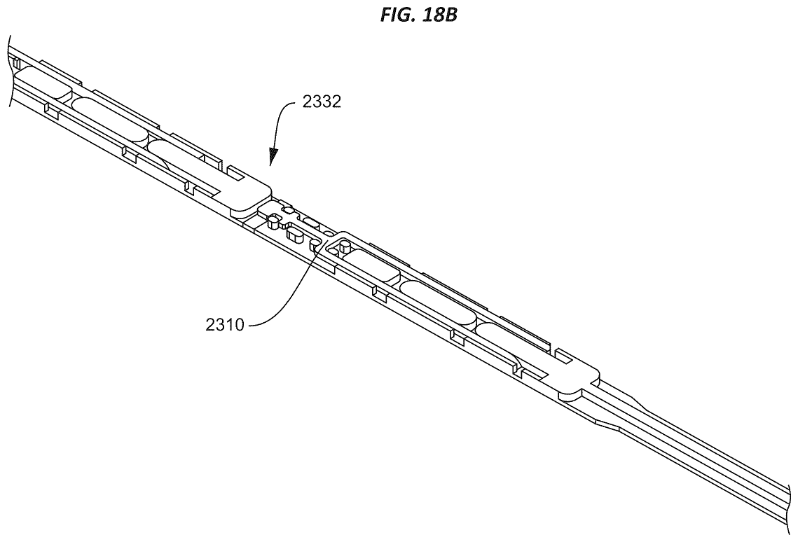

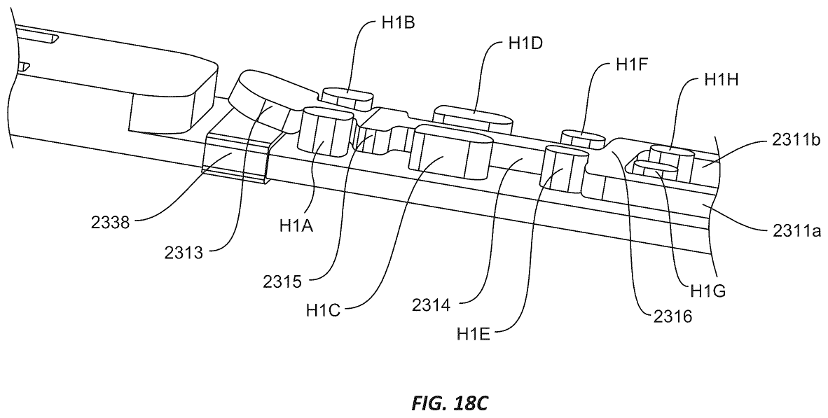

FIG. 18A is an illustration of a suture 2310 with angled tips and an engagement portion, in a partially flattened configuration, according to an embodiment. FIGS. 18B-18C are illustrations of a suture with angled tips and an engagement portion, where the suture is engaged with a SARE, according to an embodiment. As shown, the suture 2310 includes a first prong leg 2311A and a second prong leg 2311B connected via a bridging element 2316, a tail 2314, a structure 2313 (e.g., clamp head) on the end of the tail 2314, and an engagement element 2315. Both legs 2311A and 2311B may have sharp respective ends or sharpened tips 2312A and 2312B, configured to penetrate through graft and/or tissue. In some embodiments, the first prong leg 2311A, the second prong leg 2311B, the sharpened tips 2312A, 2312B, the structure 2313, the tail 2314, the engagement element 2315, and the bridging element 2316 can be the same or substantially similar to the first prong leg 2211A, the second prong leg 2211B, the sharpened tips 2312A, 2312B, the structure 2213, the tail 2214, the engagement element 2215, and the bridging element 2216, as described above with reference to FIGS. 17A-17B. Thus, certain aspects of the first prong leg 2311A, the second prong leg 2311B, the sharpened tips 2312A, 2312B, the structure 2313, the tail 2314, the engagement element 2315, and the bridging element 2316 are not described in greater detail herein.

The suture 2310 as shown in FIGS. 18A-18C is in a high energy configuration, as it is held in a substantially flat configuration, e.g., between a SARE and suture housing. The suture 2310, when held in such configuration, may not be entirely flat, given the clearance or space between the SARE and the suture housing, as well as the tendency of the suture to revert to its curved configuration. As such, the legs 2311A and 2311B of the suture may be angled upward, which can be beneficial for deployment from a window of the suture housing and subsequent penetration into the tissue.

In use, initial deployment forces are low as the sharpened tips 2312A, 2312B penetrate the graft and aorta. Once the bridging element 2316 engages the graft, the deployment forces increase significantly as the aorta and graft push back against the bridging element 2316 and subsequent deployment of the tail 2314. The suture 2310 can be deployed in a similar way as suture 2010, and therefore further details of how the suture 2310 is deployed are not described in greater detail herein.

FIGS. 18B-18C show the suture 2310 engaged with a SARE 2332, where the suture 2310 is in a flattened or substantially flattened configuration. While not depicted, it can be understood that the suture 2332 can be held in a flattened or substantially flattened configuration by the SARE 2332 and a suture housing (not depicted) disposed around the SARE 2332. The SARE 2332 includes a radiopaque marker 2338 (or be coated with a radiopaque material or be an alloy including radiopaque material at the location of the marker), and protrusions H1A, H1B, H1C, H1D, H1E, H1F, H1G, H1H. The protrusions H1A, H1B, H1C, H1D, H1E, H1F, H1G, H1H can enhance the traction of deployment of the suture 2310 and act as grips or frictional surfaces to force or push the suture 2310 out of the suture housing. Additionally, the protrusions H1A, H1B, H1C, H1D, H1E, H1F, H1G, H1H can aid in preventing the structure 2313 from changing shape within the suture housing and/or being forced into undesirable locations within the suture housing (e.g., if there is an area created by tolerances between the SARE 2332 and the housing).

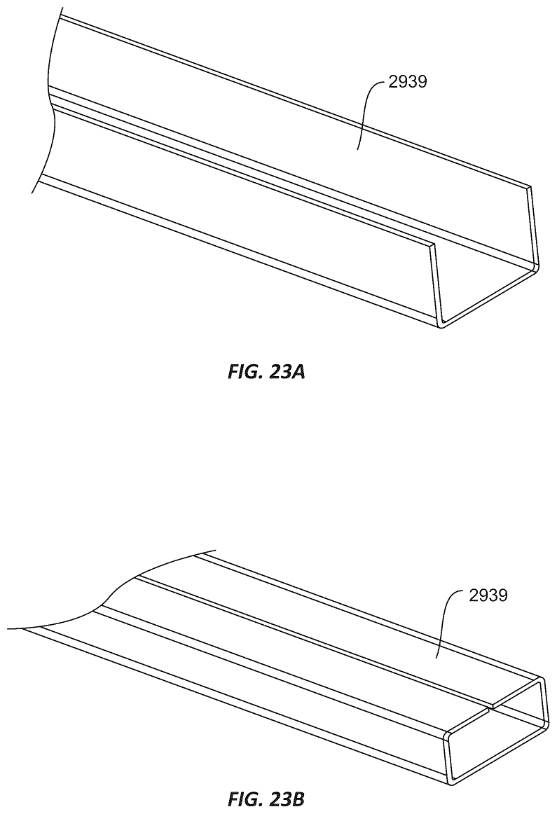

In embodiments, when the suture 2310 is loaded in the SARE 2332 and suture housing, the forces to keep the suture 2310 in a flattened condition resolve at each end of the suture 2310. The ends of the suture 2310 (i.e., the structure 2313 and the sharpened tips 2312A, 2312B) can bend or curve slightly and potentially push against the interior wall of the suture housing. These ends can slide along the interior wall of the suture housing and create friction during deployment. As the sutures deploy, the first deployment has the highest corresponding friction since deployment action moves all of the loaded sutures relative to the suture housing, and each subsequent deployed suture reduces the coefficient of friction between the sharpened tips 2312A and the structures 2313 of the sutures contained within the suture housing against the interior surface of the suture housing. As such, it can be desirable to reinforce the suture housing or use different or a combination of materials for the suture housing that can withstand such frictional forces. An example of such is described with reference to FIGS. 23A-23B, below.

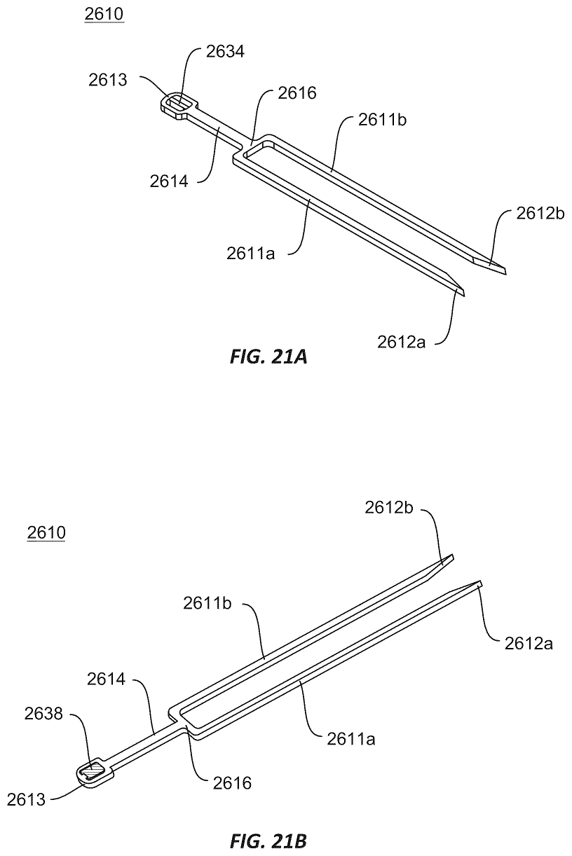

As shown, the radiopaque marker 2338 is positioned under the structure 2313, In some embodiments, the SARE 2332 can include a radiopaque marker positioned under the engagement element 2315. In some embodiments, the SARE 2332 can include a radiopaque marker positioned under the tail 2314. In some embodiments, the SARE 2332 can include a radiopaque marker positioned under the first prong leg 2311A and/or the second prong leg 2311B. The radiopaque marker 2338 can aid in tracking the movement of the SARE 2332 and/or the suture 2310. In some cases, the suture 2310 can be seen under fluoroscopy. However, making the suture 2310 more radiopaque can have advantages. For example, a radiopaque suture can further aid in visualization for positioning or for identification of the suture 2310 at a later date. Gold deposition or other suitable radiopaque materials can be used to enhance the radiopacity of the suture 2310. Further details of radiopaque sutures are provided with reference to FIGS. 21A-21B.

As shown, the protrusions H1A, H1B are positioned between the structure 2313 and the engagement element 2315 on either side of the tail 2314. The protrusions H1A, H1B are positioned such that the protrusions H1A, H1B can contact the engagement element 2315 as the suture 2310 is being advanced. In some embodiments, the protrusions H1A, H1B can contact the tail 2314. As shown, the protrusions H1C, H1D are positioned distal to the engagement element 2315 on either side of the tail 2314. In some embodiments, the protrusions H1C, H1D can contact the tail 2314 and/or the engagement element 2315. As shown, the protrusions H1E, H1F are positioned proximal to the bridging element 2316 on either side of the tail 2314. In some embodiments, the protrusions H1E, H1F can contact the bridging element 2316. In some embodiments, the protrusions H1E, H1F can contact the tail 2314. As shown, the protrusions H1G, H1H are positioned distal to the bridging element 2316 on an interior side of the prong legs 2311A, 2311B. As shown, the protrusions H1G, H1H take up only a portion of the space between the prong legs 2311A, 2311B. This can reduce the amount of material used to form the SARE 2332.

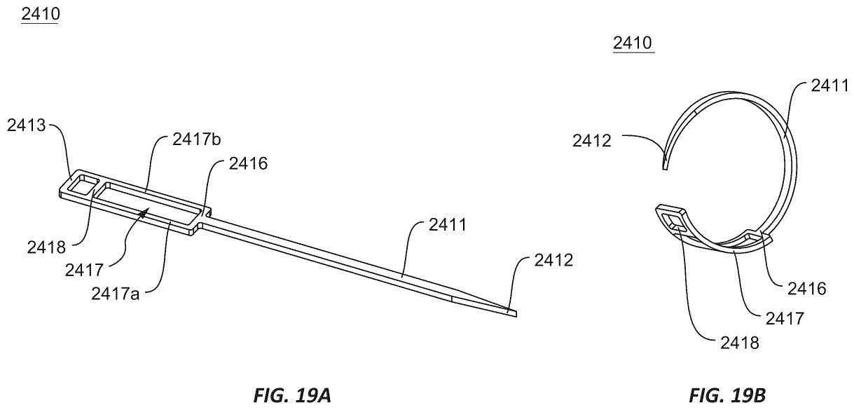

FIGS. 19A-19B show a suture 2410 with a positive deployment feature (e.g., an engagement element) and a single leg, according to an embodiment. FIG. 19A shows the suture 2410 in a flat configuration while FIG. 19B shows the suture 2410 in a curved configuration. As shown, the suture 2410 includes a prong leg 2411, a sharpened tip 2412, a structure 2413, a bridging element 2416, and a suture clamp or tail 2417 with a positive deployment feature 2418. In some embodiments, the prong leg 2411, the sharpened tip 2412, the structure 2413, and the bridging element 2416 can have the same or substantially similar properties to the prong legs 2311A, 2311B, the sharpened tips 2312A, 2312B, the structure 2313, and the bridging element 2316, as described above with reference to FIGS. 18A-18C. Thus, certain aspects of the prong leg 2411, the sharpened tip 2412, the structure 2413, and the bridging element 2416 are not described in greater detail herein.

When placing surgical grafts in the aorta, a problem often encountered involves dealing with a calcified aorta. One method often used to address such a problem is to “hunt and peck” in the area where the suture is to be placed, until a spot is found where calcium is the least dense and the suture needle moves through more easily. By employing the suture 2410 with a single prong leg 2411, this can be at least partially avoided, as there is one less prong leg that needs to have a clear path through the aorta to bind the graft and the aortic tissue together.

An obstacle encountered with single leg sutures is the difficulty of having the suture track straight during deployment. The suture 2410 can include features to improve its tracking. The prong leg 2411 can have a wider width that sutures with multiple legs (e.g., suture 2310). In some embodiments, the prong leg 2411 can have a width of about 0.5 mm, about 0.6 mm, about 0.7 mm, about 0.8 mm, about 0.9 mm, about 1 mm, about 1.1 mm, about 1.2 mm, about 1.3 mm, about 1.4 mm, or about 1.5 mm, inclusive of all values and ranges therebetween. A wider prong leg can stabilize during deployment and track straighter than a narrower prong leg. Additionally, the prong leg 2411 can have a rectangular cross section, such that the width of the prong leg 2411 is greater than the thickness of the prong leg 2411, which can also enable straighter tracking.

As shown, the suture clamp 2417 includes clamp legs 2417A, 2417B arranged in a rectangular form with a void space in the middle of the clamp legs 2417A, 2417B, the bridging element 2416, and the positive deployment feature 2418. In some embodiments, the suture clamp 2417 can mate with an appropriate receiving element in a sled (e.g., protrusions similar to the protrusions H1A, H1B, H1C, H1D, H1E, H1F, H1G, H1H, as described above with reference to FIGS. 18A-18C). As shown, the positive deployment feature 2418 is a bar that extends across the suture clamp 2417. In some embodiments, the positive deployment feature 2418 can engage with one or more protrusions on the sled (e.g., protrusions similar to the protrusions H1A, H1B, H1C, H1D, H1E, H1F, H1G, H1H, as described above with reference to FIGS. 18A-18C) to secure the suture 2410 in place on the sled. In some embodiments, the suture 2410 can have any of the curving properties of the suture 2310, as described above with reference to FIGS. 18A-18C.

FIGS. 20A-20B show a suture 2510 with a positive deployment feature (e.g., an engagement element) and two legs, according to an embodiment. FIG. 20A shows the suture 2510 in a flat configuration, while FIG. 20B shows the suture 2510 in a curved configuration. As shown, the suture 2510 includes prong legs 2511A, 2511B, sharpened tips 2512A, 2512B, a structure 2513, a bridging element 2516, and a suture clamp or tail 2517 (including clamp legs 2517A, 2517B) with a positive deployment feature 2518. In some embodiments, the prong legs 2511A, 2511B, the sharpened tips 2512A, 2512B, the structure 2513, and the bridging element 2516 can have the same or substantially similar properties to the prong legs 2311A, 2311B, the sharpened tips 2312A, 2312B, the structure 2513, and the bridging element 2316, as described above with reference to FIGS. 18A-18C. Thus, certain aspects of the prong legs 2411A, 2411B, the sharpened tips 2512A, 2512B, and the bridging element 2416 are not described in greater detail herein.

As shown, the suture 2510 has two prong legs 2511A, 2511B. In some embodiments, the prong legs 2511A, 2511B can have the same or substantially similar cross-sectional areas, such that the forces on either side of the bridging element 2516 are similar. In some embodiments, the prong leg 2511A can have a cross-sectional area greater than or less than a cross-sectional area of the prong leg 2511B, such that the forces differ on either side of the bridging element 2516. This can cause the suture 2510 to deploy in a spiral pattern as it is ejected. In some embodiments, the prong legs 2511A, 2511B can have the same or substantially similar cross-sectional areas to the clamp legs 2517A, 2517B. In some embodiments, the prong legs 2511A, 2511B can have greater or smaller cross-sectional areas than the clamp legs 2517A, 2517B. The sizing of the prong legs 2511A, 2511B and the clamp legs 2517A, 2517B is dependent on the desired path the suture 2510 is to follow upon deployment.

FIGS. 21A-21B show a suture 2610 with a radiopaque marker incorporated therein (or be coated with a radiopaque material or be an alloy including radiopaque material at the location of the marker), according to an embodiment. FIG. 21A shows the suture 2610 with a structure 2613 for receiving a radiopaque marker, and FIG. 21B shows the suture 2610 with the radiopaque marker 2638 incorporated therein. As shown, the suture 2610 includes prong legs 2611A, 2611B, sharpened tips 2612A, 2612B, a structure 2613, a tail 2614, a bridging element 2616, and a finger 2634 (e.g., elongate element) incorporated into the structure 2613. In some embodiments, the prong legs 2611A, 2611B, the sharpened tips 2612A, 2612B, the structure 2613, the tail 2614, and the bridging element 2616 can have the same or substantially similar properties to the prong legs 2211A, 2211B, the sharpened tips 2212A, 2212B, the structure 2213, the tail 2214, and the bridging element 2216, as described above with reference to FIGS. 17A-17B. Thus, certain aspects of the prong legs 2611A, 2611B, the sharpened tips 2612A, 2612B, the structure 2613, the tail 2614, and the bridging element 2616 are not described in greater detail herein.

As shown, the finger 2634 includes a column of material extending into a cavity in the structure 2613. The finger 2634 is situated, such that the radiopaque marker 2638 can be inserted into the finger 2634 or wrapped around the finger 2634. In some embodiments, the finger 2634 can be formed via machining the structure 2613. In some embodiments, the finger 2634 can be formed via laser cutting the structure 2613. In some embodiments, the finger 2634 can extend the entire length of the cavity in the structure 2613. In some embodiments, the finger 2634 can extend a portion of the length of the cavity in the structure 2613. In some embodiments, the finger 2634 can be bendable, such that it can be rotated out of plane with the structure 2613, such that a radiopaque marker 2638 can be placed thereon. In some embodiments, the radiopaque marker 2638 can have the same or substantially similar properties to the radiopaque marker 2338 as described above, with reference to FIGS. 18A-18C.

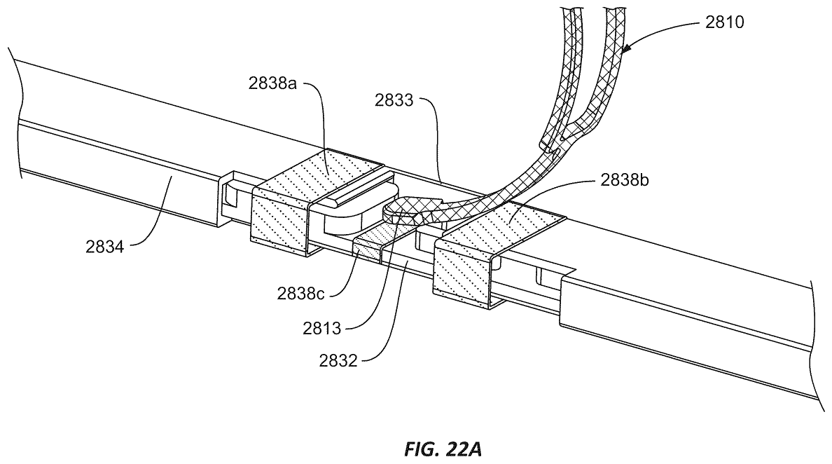

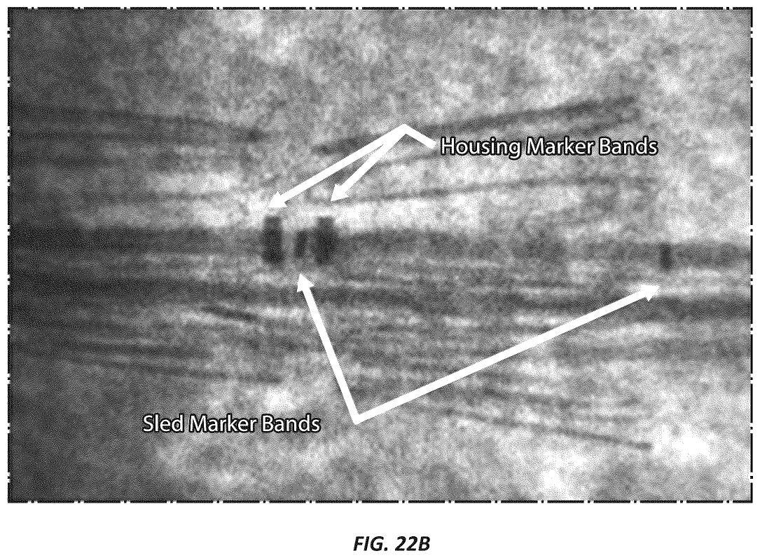

Radiopaque markers (or be coated with a radiopaque material or be an alloy including radiopaque material at the location of the marker) can also be placed on other components of a suture delivery system, including, for example, the suture housing or the SARE, FIG. 22A shows a suture 2810 exiting a suture housing 2834, according to an embodiment. As shown, the suture 2810 is being deployed from a SARE 2832. The suture housing 2834 includes a deployment window 2833 with radiopaque markers 2838A, 2838B deployed on either side of the deployment window 2833. The SARE 2832 includes a radiopaque marker 2838C placed beneath a structure 2813 of the suture 2810. In some embodiments, the suture 2810 and the structure 2813 can be the same or substantially similar to the suture 2210 and the structure 2213 as described above with reference to FIGS. 15A-15B. In some embodiments, the suture housing 2834 and the deployment window 2833 can be the same or substantially similar to the suture housing 734 and the deployment window 733, as described above with reference to FIGS. 7A-7B. In some embodiments, the SARE 2832 can be the same or substantially similar to the SARE 2332, as described above with reference to FIGS. 18A-18C, or other SAREs described herein (e.g., SARE 132, 1532, etc.). Thus, certain aspects of the suture 2810, the structure 2813, the suture housing 2834, the deployment window 2833, and the SARE 2832 are not described in greater detail herein.

There are several benefits of including the radiopaque markers 2838A, 2838B on either side of the deployment window 2833. For example, it may be desirable to determine whether a suture has been fully deployed. It may be difficult to determine if the tail and the structure 2813 are completely out of the housing window, or in a position where the suture 2810 will be completely out of the window when the biasing mesh (not shown) of the deployment system is retracted. When the biasing mesh is completely expanded, the structure 2813 can still be in the area of the deployment window 2833 and cannot be seen against the SARE 2832. As shown, the suture housing 2834 has marker bands to identify the proximal and distal limits of the deployment window 2833. As shown, the radiopaque markers 2838C is integrated into the SARE 2832 near the structure 2813, such that when the radiopaque marker 2838C is located between the radiopaque markers 2838A, 2838B, the structure 2813 is verified to be in its proper position to be released. Thus, the entire suture 2810 is fully deployed at this point and the mesh can be safely retracted.

FIG. 22B is an X-ray image of a SARE with marker bands, according to an embodiment. Two smaller radiopaque markers are visible on the SARE, and two larger radiopaque markers are included in the suture housing on either side of the window of the suture housing. As shown, one of the smaller radiopaque markers on the SARE is between the two larger radiopaque markers of the suture housing. This indicates that the structure or proximal end of a suture is aligned with the deployment window and in its proper position to be released.

Suture housings described herein can be formed of flexible material (e.g., a flexible plastic) and therefore be susceptible to wear from sutures, which can be formed of harder (e.g., metallic material). As such, suture housing can include metallic bearings, shims, or other structures that can prevent or reduce wear along a length of the distal portions of the housings containing the sutures. FIGS. 23A-23B show a manufacturing process of a shim 2939 of a suture housing, according to an embodiment. Suture housing can be constructed from a square or rectangular extrusion of a strong plastic (e.g., polyetheretherketone (PEEK)), metal (e.g., stainless steel, aluminum, or any combination thereof), or other suitable material. Stainless steel, however, has a relatively high coefficient of friction and can deform from tip forces, which increases friction as well. If the friction becomes too significant from loading and deploying sutures, the shim 2939 can distort or wrinkle. Solutions for addressing this are described in the paragraphs below. The ceiling of the housing e.g., the interior side of the housing that the suture presses against) can be reinforced with the shim 2939. In some embodiments, the shim can have a thickness of 0.01 mm, 0.015 mm, 0.02 mm, 0.025 mm, 0.03 mm, 0.035 mm, 0.04 mm, 0.045 mm, about 0.05 mm, about 0.055 mm, about 0.06 mm, about 0.065 mm, about 0.07 mm, about 0.075 mm, about 0.08 mm, about 0.085 mm, about 0.09 mm, about 0.095 mm, about 0.1 mm, inclusive of all values and ranges therebetween.

In some embodiments, the coefficient of friction of the shim 2939 can be reduced by coating the shim 2939 with a hard friction reduction coating such as, for example, a diamond-like coating (DLC). The DLC can include a hard carbon film coating that resists scratching, with a low coefficient of friction. The DLC can reduce friction of the suture tips moving along the shim 2939. In some embodiments, deformation of the shim 2939 can be prevented by making a complete housing from a heavier, thicker-walled shim (e.g., about 0.05 mm to about 0.1 mm, inclusive of all values and ranges therebetween). Deposition of DLC can be difficult in a small tube such as a square or rectangular structure (e.g., a hypotube) used for a suture housing. Therefore, a suture housing coated on one side with DLC can be formed from sheet stock (e.g., formed of stainless steel or other materials) with sequential bending, as shown in FIGS. 23A-23B. FIG. 23A shows the shim 2939 forming via two initial bends near the center of the material sheet. FIG. 23B shows two additional bends to form the shim 2939 into a rectangular prism shape. In some embodiments, the angles formed by the bends of the shim 2939 can be about 90 degrees. In some embodiments, the angles formed by the bends of the shim 2939 can be about 60 degrees, about 70 degrees, about 80 degrees, about 90 degrees, about 100 degrees, about 110 degrees, or about 120 degrees, inclusive of all values and ranges therebetween. An advantage of a suture housing formed from a heavier, thicker-walled shim coated with DLC or similar hard, low friction coating is the easier manufacturing of a one-piece housing. Additionally, the shim 2939 is more robust when thicker, and does not distort or wrinkle from deploying multiple sutures.

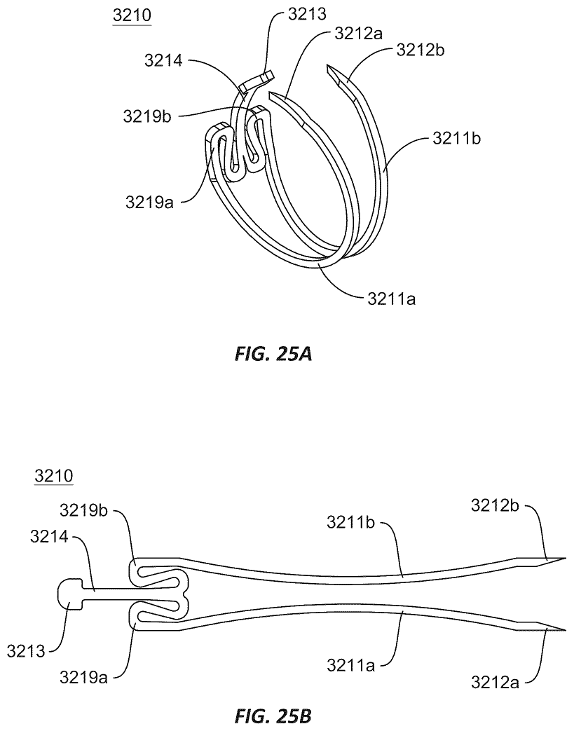

FIG. 25A-25B are illustrations of a suture 3210 including curves 3219A and 3219B in a direction orthogonal to the radius of curvature, according to embodiments. The suture 3210 can be structurally and/or functionally similar to other sutures described herein. FIG. 25A shows the suture 3210 in a curved configuration, while FIG. 25B shows the suture 3210 in a flat configuration. As shown, the suture 3210 includes prong legs 3211A, 3211B, sharpened tips 3212A, 3212B, tail 3214, and clamp head 3213. In some embodiments, the prong legs 3211A, 3211B, the bridging element 3216, the sharpened tips 3212A, 3212B, the tail 3214, and the clamp head 3213 can have the same or substantially similar properties to the prong legs 3011A, 3011B, the bridging element 3016, the sharpened tips 3012A, 3012B, the tail 3014, and the clamp head 3013, as described above with reference to FIG. 24A. Thus, certain aspects of the prong legs 3211A, 3211B, the sharpened tips 3212A, 3212B, and the bridging element 3216 are not described in greater detail herein.

As shown, the suture 3210 has curves implemented as “S” shape curves 3219A, 3219B. The “S” shape curves 3219A, 3219B can be disposed between the two prong legs 3211A, 3211B and the tail 3214. Similar to the curves or bends 3019 described above, the “S” shape curve 3219A, 3219B can be configured to increase the effective length of the suture and enable the suture to have a smaller diameter without exceeding its elastic limit when flattened. Additionally, the prong legs 3211A, 3211B can have a slight inward bend or curve between the sharpened tips 3212A, 3212B and the “S” shaped curves 3219A, 3219B. The slight inward bend of the prong legs 3211A, 3211B can be structurally and/or functionally similar to that of the slight inward bends described above with reference to FIG. 24A-B. For example, the slight inward bend or curve of the prong legs 3211A, 3211B can provide higher or improved retention of the suture in the tissue after implantation.

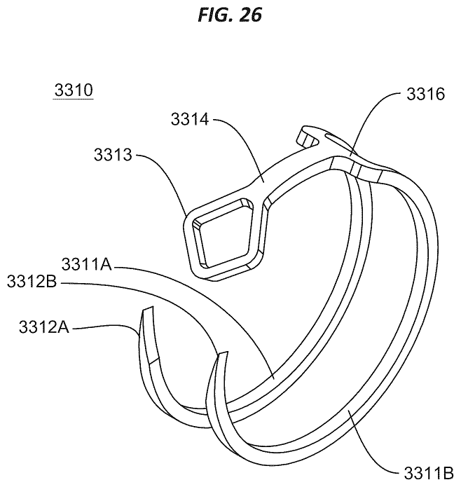

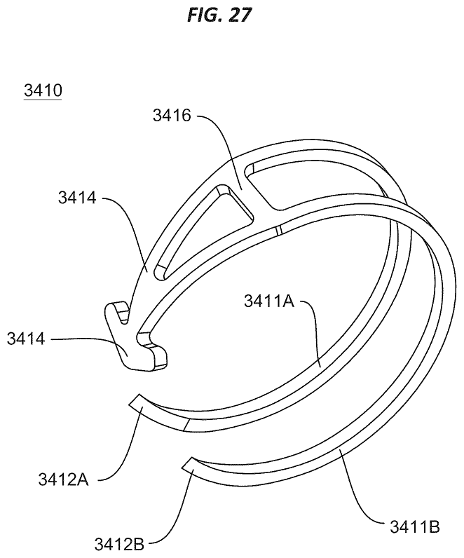

FIG. 26 is an illustration of a suture 3310 in a curved configuration including a clamp head 3313 designed to reduce interference from external devices, according to embodiments. The suture 3310 can be structurally and/or functionally similar to other sutures described herein. For example, the suture 3310 includes prong legs 3311A, 3311B, sharpened tips 3312A, 3312B, tail 3314, a bridging element 3316, and clamp head 3313. In some embodiments, the prong legs 3311A, 3311B, the sharpened tips 3312A, 3312B, the tail 3314, and the bridging element 3316 can have the same or substantially similar properties to the prong legs 3011A, 3011B, the sharpened tips 3012A, 3012B, the tail 3014, and the bridging element 3014, as described above with reference to FIG. 24A. Thus, certain aspects of the prong legs 3311A, 3311B, the sharpened tips 3312A, 3312B, the tail 3014, and the bridging element 3316 are not described in greater detail herein.

As shown, the suture 3310 includes a clamp head 3313 having a kite shape and defining an opening. Similar to the clamp head 3013 described above, the clamp head 3313 can include smooth angles to guide an external device away from the clamp head 3313 and reduce likelihood of an external device catching on clam head 3313 and disturbing the suture 3310.

FIG. 27 is an illustration of a suture 3410 in a curved configuration with an arrowhead-shaped clamp head 3413, according to embodiments. The suture 3410 can be structurally and/or functionally similar to other sutures described herein. For example, the suture 3410 includes two prong legs 3411A, 3411B, sharpened tips 3412A, 3412B, and a bridging element 3416 that can have the same or substantially similar properties to the prong legs 3011A, 3011B, the sharpened tips 3012A, 3012B, and the bridging element 3016, as described above with reference to FIG. 24A. Thus, certain aspects of the prong legs 3411A, 3411B, the sharpened tips 3412A, 3412B, and the bridging element 3416 are not described in greater detail herein.

As shown, suture 3410 includes a clamp head 3413 having an arrowhead shape with smooth edges. The arrowhead-shaped clamp head 3413 can guide an external device towards the top of the clamp head 3413 and away from the suture 3410, thereby reducing risk of the external device disturbing or dislodging the suture 3410.

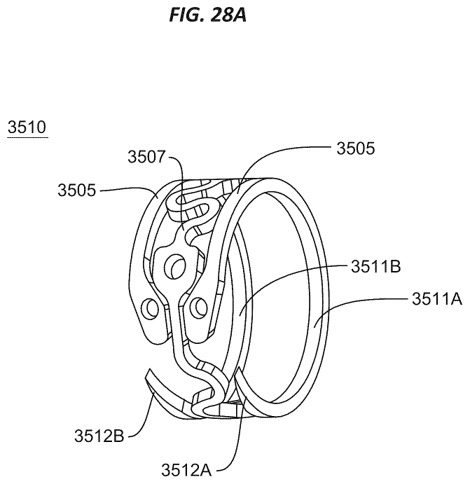

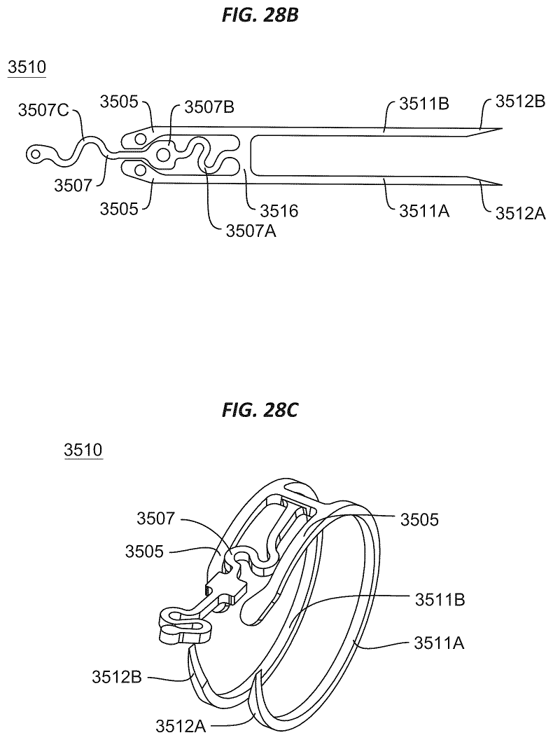

FIG. 28A-28C are illustrations of a suture 3510 including two clamp arms 3505 and a protective shield 3507, according to embodiments. The suture 3510 can be structurally and/or functionally similar to other sutures described herein. For example, the suture 3510 includes two prong legs 3511A, 3511B, sharpened tips 3512A, 3512B, and a bridging element 3516 that can have the same or substantially similar properties to the prong legs 3011A, 3011B, the sharpened tips 3012A, 3012B, and the bridging element 3016, as described above with reference to FIG. 24A. Additionally, the suture 3510 includes two clamp arms 3505 extending parallel from one another from the bridging element 3516. A protective shield 3507 is disposed between the two clamp arms 3505 and extends a longer distance from the bridging element than the two clamp arms 3505. The protective shield 3507 includes a first “S” shaped curve 3507a disposed in a channel or space between the clamp arms 3505. The first “S” shaped curve 3507a connects to a protective tab 3507b disposed between the ends of the clamp arms 3505. The protective tab connects to an elongated tail that extends beyond the clamp arms 3505 and forms a second “S” shaped curve 3507c. The protective shield 3507 is structurally and/or functionality similar to that of the protective shield 3107 described above with reference to FIG. 24B. For example, the protective shield 3507 can be configured to may flex or bend away from an external device if the external device moves under the tip of the shield itself, thereby reducing risk of disturbing the suture 3510. Prior to loading and/or use, the first S-shape can be pulled to yield (i.e., pulled beyond its yield point) such that the protective tab, after the suture is deployed and in its curved configuration, is configured to sit on top of the clamp arms 3405 and cover the channel between the clamp arms.

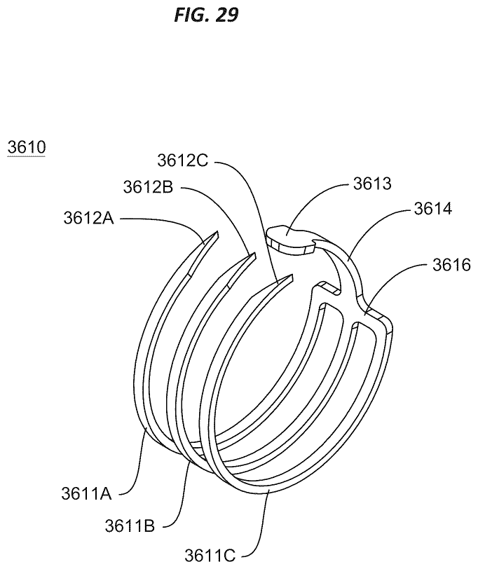

FIG. 29 is an illustration of a suture 3610 in a curved configuration including three prong legs 3611A, 3611B, 3611C, according to embodiments. The suture 3610 can be structurally and/or functionally similar to other sutures described herein. For example, the suture has prong legs 3611A, 3611B, 3611C, a bridging element 3616, a tail 3614, and a clamp head 3613 that can have the same or substantially similar properties to the prong legs 3011A-3011N, the sharpened tips 3012A-3012N, the bridging element 3016, the tail 3014, and the clamp head 3013 as described above with reference to FIG. 24A. As shown, prong legs 3611A, 3611B, 3611C are evenly spaced along the bridging element 3616. However, it can be appreciated that the sutures described herein can have any distribution of prong legs 3611A, 3611B, 3611C. Prong leg 3611B is in line with tail 3613 and extends from the bridging element 3616 away from tail 3613. The additional prong legs provides additional holding power or retention of the suture 3610.

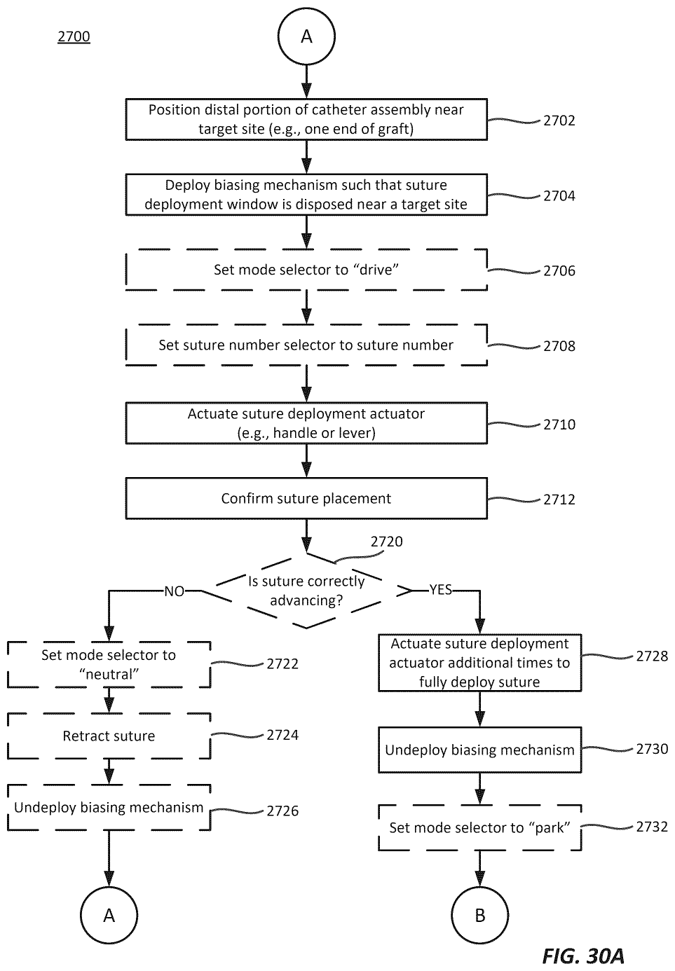

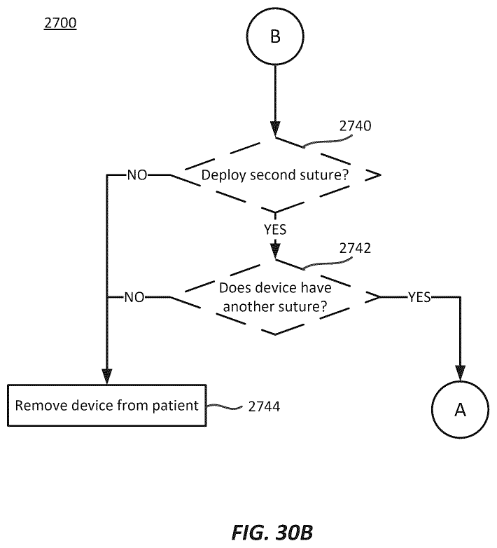

FIG. 30A shows an example process 2700 of deploying sutures consistent with a disclosed embodiment. At step 2702 of process 2700, a distal portion of a catheter assembly is positioned near a target site (e.g., near a wall of a graft, or a surface of a tissue that requires suturing). At step 2704, a device for deploying sutures (e.g., device 700, as shown in FIG. 6A) is manipulated (e.g., by a surgeon) to deploy a biasing mechanism such that a suture deployment window located in suture housing is disposed near a target site. The biasing mechanism is configured to expand within a cavity in which the catheter assembly is placed, and is configured to press at least a portion of the suture housing, having the suture deployment window, to a surface of a tissue (e.g., a graft, an aorta, and the like) that requires suturing. At step 2706, a surgeon operating the device is configured to set a mode selector of a mode selection system to “drive” (mode “D”) as described above. At step 2708, the surgeon is configured to set a suture number selection mechanism to a particular suture number to deploy a particular suture, as described above, and at step 2710, actuate a suture deployment actuator by manipulating a suture deployment control mechanism (e.g., handle or lever) to initiate deployment of a suture. In an example embodiment, after partially deploying the suture, the surgeon may confirm a suture placement at step 2712. The confirmation for the suture placement may be accomplished using any suitable means (e.g., using imaging such as internal camera, CT scan, ultrasound, and the like, as described above).

If the suture is correctly placed (step 2720, Yes), at step 2728, the suture deployment actuator may be actuated several additional times (e.g., a suture deployment control mechanism 743, as shown in FIG. 8A, may be pressed several times) for the suture to be fully deployed. At step 2730, a biasing mechanism is undeployed (e.g., when the biasing mechanism is an expandable mesh, the mesh may be fully contracted). In an example embodiment, the biasing mechanism is undeployed by actuating a biasing mechanism actuator, as described above. At step 2732, the surgeon may set the mode selector into “park” mode (“P” mode) as described above. In park mode, the suture deployment control mechanism (e.g., the suture deployment control mechanism, as described above) is fixed, and no new sutures may be deployed.

If the suture is not correctly placed (step 2720, No), at step 2722 the surgeon may set the mode selector into “neutral” mode (“N” mode) as described above. By setting mode selector into the neutral configuration, the surgeon may slide a SARE (e.g., SARE 132) towards a distal end of device 700, and, as a result, at step 2724, retract the suture back into a suture housing. At step 2726, the surgeon may operate the suture delivery device and undeploy the biasing mechanism. Step 2726 may be the same as step 2730, as described above. After undeploying the biasing mechanism, the surgent may move a catheter assembly of a device (e.g., catheter assembly 730 of device 700) to a new site for performing suturing at that site. After moving to the new site, process 2700 may be repeated.

FIG. 30B shows a further set of steps of process 2700 that may be performed after step 2732. At step 2740, if a second suture needs to be deployed (step 2740, Yes), the surgeon may determine if SARE contains more sutures. If SARE contains more sutures (step 2742, Yes), process 2700 may be repeated starting with step 2702. Alternatively, if SARE does not contain more sutures (step 2742, No), at step 2744, catheter assembly 730 may be removed from a body of a patient (e.g., entire device 700 may be withdrawn, and, in some cases, be reloaded with a new catheter assembly, or a new SARE containing sutures may be changed). At step 2740, if a second suture does not need to be deployed (step 2740, No), step 2744 is performed as described above.

It should be understood that the examples and illustrations in this disclosure serve exemplary purposes and departures and variations in shapes of elements (e.g., sutures), and operation of various components (e.g., operation of biasing member), may be varied according to the teachings herein without departing from the scope of this invention.

As used herein, the terms “about” and/or “approximately” and/or “substantially” when used in conjunction with numerical values and/or ranges generally refer to those numerical values and/or ranges near to a recited numerical value and/or range. In some instances, the terms “about” and “approximately” and “substantially” may mean within ±10% of the recited value. For example, in some instances, “about 100 [units]” may mean within ±10% of 100 (e.g., from 90 to 110). The terms “about” and “approximately” may be used interchangeably.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A suture, comprising:
a set of legs having proximal ends joined to each other and elongate bodies that extend parallel to one another such that the set of legs form a U-shaped structure, the set of legs having distal ends that terminate in sharpened tips that are configured to penetrate through a portion of a graft and a vessel wall adjacent to the portion of the graft;
a tail having a proximal end and a distal end, the distal end being coupled to the U-shaped structure;
a clamp head coupled to the proximal end of the tail; and
an engagement element disposed between the U-shaped structure and the clamp head, the engagement element configured to provide a surface for engaging with a suture advancement element to deploy the suture,
the suture configured to transition from a flattened configuration in which the tail extends in a direction opposite to the direction of the elongate bodies of the legs to a curved configuration in which the tail and the set of legs are curved,
the set of legs and the tail configured to exert forces in opposite directions when the suture is in the curved configuration such that the set of legs and the tail are configured to hold the portion of the graft and the vessel wall relative to one another.

2. The suture of claim 1, wherein the suture is configured to be held within a housing in the flattened configuration and is configured to automatically transition into the curved configuration when released from the housing.

3. The suture of claim 2, wherein the suture is configured to be released from the housing via an opening in the housing with the sharpened tips of the suture exiting the housing first.

4. The suture of claim 3, wherein the sharpened tips of the suture are configured to curve and to penetrate through the portion of the graft and the vessel wall while exiting from the housing.

5. The suture of claim 1, wherein the suture in the curved configuration is configured to form an annular loop.

6. The suture of claim 5, wherein at least a portion of the tail is configured to be disposed within an outer perimeter of the annular loop when the suture is in the curved configuration.

7. The suture of claim 5, wherein the set of legs and the tail are configured to be disposed along an outer perimeter of the annular loop when the suture is in the curved configuration.

8. The suture of claim 5, wherein at least a portion of the tail is configured to be disposed outside of an outer perimeter of the annular loop when the suture is in the curved configuration.

9. The suture of claim 1, wherein the tail of the suture is configured to press against the portion of the graft that is disposed between at least a portion of the set of legs when the suture is in the curved configuration and positioned to hold the portion of the graft and the vessel wall relative to one another.

10. The suture of claim 1, wherein the suture is formed from a superelastic material.

11. The suture of claim 1, wherein the suture is formed by laser cutting a metallic tube.

12. The suture of claim 1, wherein the suture is formed by laser cutting a portion of a metallic tube and electropolishing one or more edges of the cut portion to round them.

13. The suture of claim 1, wherein the set of legs and the tail are configured to generate a clamping or tightening force when the suture is in the curved configuration.

14. The suture of claim 1, wherein the suture includes a radiopaque marker, a coating of radiopaque material, or a nitinol alloy including a radiopaque material.

15. A suture, comprising:
a single leg having an elongate body, the single leg having a distal end that terminates in a sharpened tip that is configured to penetrate through a portion of a graft and a vessel wall adjacent to the portion of the graft;
a clamp having a proximal end and a distal end, the distal end being coupled to the single leg; and
a clamp head coupled to the proximal end of the clamp,
the suture configured to transition from a flattened configuration in which the clamp extends in a direction opposite to the direction of the elongate body of the single leg to a curved configuration in which the clamp and the single leg are curved,
the single leg and the clamp configured to exert forces in opposite directions when the suture is in the curved configuration such that the single leg and the clamp are configured to hold the portion of the graft and the vessel wall relative to one another.

16. The suture of claim 15, wherein the single leg has a cross-section with a first lateral dimension that is greater than a second lateral dimension.

17. The suture of claim 16, wherein the cross-section is a rectangular cross-section.

18. The suture of claim 15, wherein the clamp has a width that is greater than a width of the single leg.

19. The suture of claim 15, further comprising:
an engagement element disposed at a point along a length of the clamp, the engagement element configured to provide a surface for engaging with a suture advancement element to deploy the suture.

20. The suture of claim 15, wherein the suture is configured to be held within a housing in the flattened configuration and is configured to automatically transition into the curved configuration when released from the housing.

21.-38 (canceled)
